(19) United States
(12) Patent Application Publication
(10) Pub. No.: US 2011/0195407 A1
(43) Pub. Date: Aug. 11, 2011

(54) NUCLEIC ACID OCCURRING IN BLOOD FROM HEPATITIS PATIENT, POLYPEPTIDE ENCODED BY THE NUCLEIC ACID, AND USES OF THE SAME

(75) Inventors: Kouei Satoh, Koga-shi (JP); Akiko Takakura, Tokyo (JP)

(73) Assignee: Japanese Red Cross Society, Minato-ku, Tokyo (JP)

(21) Appl. No.: 13/057,040

(22) PCT Filed: Aug. 3, 2009
(86) PCT No.: PCT/JP2009/063766

§ 371 Date: Mar. 31, 2011

(30) Foreign Application Priority Data
Aug. 1, 2008 (JP) 2008-200050

(51) Int. Cl. C12Q 1/68; C07H 21/00; C07K 14/00; C12N 15/63; C12N 1/00; C12P 21/00; C07K 16/00

(52) U.S. Cl. 435/6.1; 536/23.1; 530/326; 530/325; 530/324; 435/320.1; 435/243; 435/69.1; 530/387.9

(57) ABSTRACT

A substance associated with cryptogenic hepatitis is found and information, particularly genetic information, on the substance is provided. Also, a method for detecting the substance and a material which can be used in the method are provided. A nucleic acid is extracted from each of 500 blood samples which have alanine aminotransferase (ALT) abnormality and are negative for a hepatitis virus marker, and the amplification of the nucleic acid is carried out by using helicase family-reactive helicase primers which have been produced uniquely. As a result, a novel nucleotide sequence (SEQ ID NO:1) can be obtained, and it is found that the nucleotide sequence occurs at high frequency in cryptogenic hepatitis.

### TECHNICAL FIELD

The present invention relates to a novel nucleic acid found in blood of a hepatitis patient and a polypeptide encoded by the same. More specifically, the present invention relates a novel nucleic acid found out to exist at a high probability in a clinical example of hepatitis in which infection with any known hepatitis virus is denied (hereinafter referred to as “hepatitis of unknown cause”), and a polypeptide encoded by the same. The present invention further relates to the use of the novel nucleic acid and the polypeptide encoded by the same.

### BACKGROUND ART

Hepatitis is an inflammation caused in liver on account of various causes, and hepatitis is a medically important disease in which the liver function is damaged sometimes resulting in the death. Those known as the cause of hepatitis include, for example, viruses, drugs, alcohol, and autoimmunity. In particular, a large number of patients exist in relation to viral hepatitis which is caused by a virus (hepatitis virus), and viral hepatitis is especially important because viral hepatitis is infected via blood and/or feces. Those having been reported until now as the hepatitis virus include those of the types of A, B, C, D, E, F, G, and TT. The relationship with hepatitis has been established in relation to the hepatitis viruses of the A, B, C, D, and E types. Many studies have been performed for these viruses. Serological and/or genetic detecting methods have been established therefor, which are widely used for the screening and the diagnosis. In particular, the screening is carried out in many countries for the transfusion blood in relation to the B type and C type hepatitis viruses which are hepatitis viruses infectious via blood, which is useful to prevent the infection with the B type hepatitis and the C type hepatitis caused by the blood transfusion.

However, the hepatitis, for which the cause or etiology has not been elucidated yet, is present (Non-Patent Documents 1 and 2). It is demanded to elucidate the cause of such hepatitis and establish a screening method, a diagnosis method, and a medical treatment method for such hepatitis.

### CITATION LIST

### Non-Patent Documents

Non-Patent Document 1: World J Gastroenterol. 2006 Aug. 21; 12(31): 5048-50;
Non-Patent Document 2: Transplantation. 2000 Jul. 27; 70(2): 292-7.

### SUMMARY OF THE INVENTION

### Problem to be Solved by the Invention

An object of the present invention is to found out a substance relevant to the hepatitis of unknown cause and provide information in relation thereto, especially gene information. Another object of the present invention is to provide a method for detecting the substance and a material usable for the method.

### Solution to Problem

The present inventors have extracted nucleic acids from 500 blood samples which are abnormal in alanine aminotransferase (ALT)/negative in hepatitis virus marker, and the present inventors have performed the amplification by using a uniquely prepared helicase family-reactive helicase primer. As a result, it has been found out that the amplification is observed for the nucleic acids of 12 samples. Sequences of the amplified nucleic acids are determined to perform the retrieval by using the nucleic acid sequence database. As a result, 8 samples are coincident with known nucleic acid sequences. However, any coincident nucleic acid sequence is not found out in the database in relation to 4 samples. The sequences of 4 samples are identical with each other. The present inventors have carried out the primer walking by using the sample in which the novel sequence has been found out, and the present inventors have further clarified sequences in the 3′ direction and the 5′ direction of the gene including the novel sequence. Finally, a novel nucleic acid sequence (SEQ ID NO: 1) having a length of 9496 nucleotides has been obtained. As a result of the database retrieval, it has been confirmed that the obtained nucleic acid sequence is a novel sequence.

A method for detecting the presence of the concerning novel gene has been completed on the basis of the sequence obtained as described above. Further, the analysis has been performed for the sample, for example, in relation to the possibility/impossibility of reverse transcription, the sensitivity to RNase and DNase, and the restriction enzyme reaction. As a result, it has been revealed that this nucleic acid sequence exists as double strand DNA. According to the structural analysis for the double strand DNA thus revealed, estimated open reading frame regions (SEQ ID NOS: 3 to 9) have been specified. Amino acid sequences (SEQ ID NOS: 10 to 17), which are encoded by these regions, have been revealed. Further, concerning novel DNA's are obtained for various clinical specimens, and nucleotide sequences thereof are investigated in detail. As a result, it has been revealed that the mutation occurs in the gene relatively scarcely, and the sequence is conserved. An oligonucleotide, which is useful to detect the concerning gene, has been successfully designed.

The present invention has been completed on the basis of the knowledge as described above, which includes the following inventions.

(1) A nucleic acid comprising a nucleotide sequence of SEQ ID NO: 1.

(2) A nucleic acid comprising a nucleotide sequence complementary to a nucleotide sequence of SEQ ID NO: 1.

(3) A nucleic acid comprising a nucleotide sequence of an open reading frame encoding a protein having a length of not less than 20 amino acids in a nucleotide sequence of SEQ ID NO: 1.

(4) A polypeptide comprising an amino acid sequence encoded by the open reading frame of the nucleic acid or an amino acid sequence of a fragment thereof having a length of not less than 20 amino acids.

(5) A polynucleotide which has a nucleotide length of not less than 10 bases, which includes a partial nucleotide sequence of a nucleotide sequence of SEQ ID NO: 1 or a nucleotide sequence complementary to the nucleotide sequence, and which is specifically hybridizable to the nucleic acid.

(6) The polynucleotide as described above, wherein the polynucleotide has a nucleotide length of not more than 50 bases.

(7) The polynucleotide as described above, wherein the polynucleotide includes any one of nucleotide sequences of SEQ ID NOS: 18 to 38.

(8) A method for detecting a nucleic acid including a nucleotide sequence of SEQ ID NO: 1, a sequence complementary thereto, or a partial sequence of any one thereof, the method comprising using one or more of the polynucleotides, as a primer or primers and/or a probe or probes.

(9) The method as described above, further comprising amplifying the nucleic acid by using the polynucleotide or polynucleotides as the primer or primers.

(10) The method as described above, wherein a combination of the polynucleotides to be used as the primers is any one of combinations of SEQ ID NOS: 18 and 19, SEQ ID NOS: 19 and 20, SEQ ID NOS: 21 and 22, SEQ ID NOS: 23 and 24, SEQ ID NOS: 25 and 26, SEQ ID NOS: 27 and 28, SEQ ID NOS: 29 and 30, SEQ ID NOS: 31 and 32, SEQ ID NOS: 33 and 34, SEQ ID NOS: 35 and 36, and SEQ ID NOS: 37 and 38.

(11) The method as described above, further comprising performing first amplification as a first stage by using a combination of SEQ ID NOS: 18 and 19 as a primer combination, and subsequently performing second amplification for a product of the amplification by using a combination of SEQ ID NOS: 19 and 20 as a primer combination.

(12) An expression vector comprising the nucleic acid as described above.

(13) A cell which is transformed by introducing the nucleic acid as described above and which expresses a polypeptide encoded by the nucleic acid.

(14) A method for producing the polypeptide, comprising culturing the cell as described above and recovering the polypeptide from a culture.

(15) An antibody against the polypeptide.

In this specification, the term “nucleic acid” is used interchangeably with “gene”, “DNA”, “polynucleotide”, and “oligonucleotide” in some cases. That is, the “nucleic acid” refers to the general nucleic acid which includes DNA and RNA. The term “polypeptide” is used interchangeably with “protein” and “peptide”.

The term “hepatitis of unknown cause” refers to the hepatitis for which the infection with any known hepatitis virus is denied. More specifically, the term “hepatitis of unknown cause” refers to the hepatitis which is negative for the virus markers for the A type, B type, and C type hepatitis viruses as main causes of hepatitises although it is clinically diagnosed that any hepatitis occurs.

### Effect of the Invention

According to the present invention, the nucleic acid relevant to the hepatitis of unknown cause is provided. The detection of the nucleic acid of the present invention and a translation product thereof is useful to diagnose the hepatitis of unknown cause.

### BRIEF DESCRIPTION OF THE DRAWINGS

FIG. 1 shows sequences of helicase family primers used to perform the screening for unknown genes and positions for setting the primers in Example.

FIG. 2 shows a sequence extending method used to determine a sequence of an unknown gene in Example.

FIG. 3 shows a procedure of a nucleic acid measuring method in Example.

FIG. 4 shows an arrangement of deduced ORF's in a nucleotide sequence of SEQ ID NO: 1.

FIG. 5 shows the construction of an expression construct.

### DESCRIPTION OF EMBODIMENTS

The nucleic acid of the present invention is found out from blood of a patient of hepatitis which has ALT abnormality and/or which is negative for the hepatitis virus marker, i.e., hepatitis of unknown cause or unknown etiology. The nucleic acid of the present invention is composed of the nucleotide sequence of SEQ ID NO: 1 or the nucleotide sequence complementary thereto. The nucleic acid of the present invention is any one of the double strand and the single strand.

The followings are achieved on the basis of the nucleotide sequence clarified by the present invention. That is, it is possible to seek out an open reading frame candidate thereof, it is possible to predict sequences of proteins encoded by structural and nonstructural gene candidates encoded by the novel gene on the basis of the information thereof, and it is possible to synthesize the protein or proteins (hereinafter referred to as “gene product” or “gene products”) encoded by the structural and/or nonstructural gene candidate or candidates and a fragment or fragments thereof chemically or by means of the genetic engineering. The method for seeking out the open reading frame candidate from the sequence and the method for expressing the gene product encoded by the gene and the fragment thereof by means of the genetic engineering or chemically synthesizing the same are well-known in the concerning technical field. Therefore, the present invention also includes the fragment of the nucleic acid, the structural and nonstructural genes included in the nucleic acid, and the gene products thereof.

The structural and nonstructural genes are exemplified by a nucleic acid composed of a nucleotide sequence of an open reading frame encoding a protein having a length of not less than 20 amino acids in the nucleotide sequence of SEQ ID NO: 1. The gene product is exemplified by a polypeptide composed of an amino acid sequence encoded by the open reading frame of the nucleic acid or an amino acid sequence of a fragment thereof having a length of not less than 20 amino acids. The length of not less than 20 amino acids is usually a length sufficient to exhibit the antigenicity.

More specifically, the structural and nonstructural genes are exemplified by a nucleic acid composed of any one of nucleotide sequences of SEQ ID NOS: 2 to 9. The gene product is exemplified by a polypeptide composed of any one of amino acid sequences of SEQ ID NOS: 10 to 17.

It is known that any mutation, which does not affects the function, may exists in the gene. Therefore, it is acknowledged that the nucleic acid or the like of the present invention includes not only those having the sequences exemplified by way of example but also those composed of nucleotide sequences and amino acid sequences in the substantially same range. The substantially same range can be judged by the ratio of the homology of the sequence distinctly distinguishable when the concerning nucleotide sequence or the amino acid sequence is compared with another sequence (for example, any known gene of hepatitis cause). The substantially same range can be determined with reference to the diversity of the sequence in a species known in relation to known organisms of the same species. For example, it is possible to clarify the taxonomical position of an organism from which the gene originates, by comparing the novel gene sequence with known gene sequences. The comparison between the novel gene and known gene sequences can be easily carried out by utilizing the database (for example, AAA) available in public.

The substantially same nucleotide sequence, which is substantially the same as the nucleotide sequence (for example, nucleotide sequences described in SEQ ID NOS: 1 to 9) of the open reading frame encoding the protein having the length of not less than 20 amino acids in relation to the nucleotide sequence of SEQ ID NO: 1, is, for example, a sequence having the identity of not less than about 95% preferably not less than 97% with respect to any one of the nucleotide sequences described above.

Further, nucleotide sequences, which code for the amino acid sequences of SEQ ID NOS: 10 to 17 encoded by the nucleotide sequences described above (for example, nucleotide sequences of SEQ ID NOS: 2 to 9) respectively in accordance with the degeneration of the genetic code, are also included in the substantially same nucleotide sequence.

The amino acid sequence, which is substantially the same as the amino acid sequence encoded by the open reading frame of the nucleic acid or the amino acid sequence of the fragment thereof having the length of not less than 20 amino acids (for example, amino acid sequences described in SEQ ID NOS: 10 to 17), is, for example, a sequence in which any amino acid in the sequence is substituted by the substitution between amino acids known not to greatly change the structure of the protein. The substitution as described above usually includes the interchangeable substitution between aliphatic amino acids Ala, Val, Leu, and Ile, the interchangeable substitution between Ser and Thr, the interchangeable substitution between Asp and Glu, the interchangeable substitution between Asn and Gln, the interchangeable substitution between Lys and Arg, and the interchangeable substitution between Phe and Tyr.

Owing to the provision of the nucleic acid of the present invention, it is possible to find out sequences specific to the nucleic acid of the present invention (especially the novel gene included in the nucleic acid) from the sequence, it is possible to synthesize oligonucleotides having the sequences and/or sequences complementary thereto, and it is also possible to detect and/or amplify the nucleic acid of the present invention by using the same as primers and/or probes. The region, which is specific to the sequence, can be easily found out in practice by utilizing the database as described above and any commercially available software (for example, Lasergene DNASTAR (trade name)). Further, it is also possible to specify the primer and/or probe suitable for the detection and/or the amplification of the gene by using any software in the same manner as described above. The method for constructing a gene detecting system by using the primer and/or probe as described above is well-known in the concerning technical field. Reference may be made, for example, to “PCR Modern Utilization Manual (written by Hiromi Sasaki, Yodo-sha, 2003). In the present invention, the polymerase chain reaction (Polymerase Chain Reaction: PCR) is used as the method for amplifying the gene by way of example. However, it is also possible to use any amplification method other than PCR, for example, the LCR (Ligase Chain Reaction) method. The two-stage PCR is also included in PCR.

The primer/probe as described above is exemplified by a polynucleotide which has a nucleotide length of not less than 10 bases, preferably not less than 15 bases, and especially preferably not less than 20 bases, which includes a partial nucleotide sequence of the nucleotide sequence of SEQ ID NO: 1 or any nucleotide sequence complementary to the nucleotide sequence, and which is specifically hybridizable to the nucleic acid of the present invention. The condition of the hybridization may be any known condition. However, the condition is usually a hybridization condition usable in a protocol of the typical southern blotting as follows.

1. DNA is transferred from a gel after agarose gel electrophoresis, and nitrocellulose, on which DNA is immobilized, is immersed in a 3×SSC solution to perform a treatment at 65° C. for 30 minutes.

2. The sample is immersed in a 5×SSC, 1% SDS, 1×denhardt solution (containing labeled probe) to perform a treatment at 65° C. for 2 hours.

3. The temperature is further held at 65° C. overnight.

4. The sample is immersed in about 100 mL of a 2×SSC, 0.1% SDS solution, followed by being permeated at room temperature.

5. The solution is exchanged with a 0.2×SSC, 0.1% SDS solution, the temperature is held at 65° C. for 30 minutes, and the solution is exchanged again to hold the temperature.

6. The sample is washed with 2×SSC and dried in air, followed by autoradiography.

The polynucleotide described above usually has a nucleotide length of not more than 50 bases, preferably not more than 40 bases, and especially preferably not more than 30 bases.

Examples of the polynucleotide described above are specifically exemplified by those including the nucleotide sequences of SEQ ID NOS: 18 to 38.

Examples of the primer combination for PCR are exemplified by SEQ ID NOS: 18 and 19, SEQ ID NOS: 19 and 20. SEQ ID NOS: 22 and 23, SEQ ID NOS: 24 and 25, SEQ ID NOS: 26 and 27, SEQ ID NOS: 28 and 29, SEQ ID NOS: 30 and 31, SEQ ID NOS: 32 and 33, SEQ ID NOS: 34 and 35, SEQ ID NOS: 36 and 37, and SEQ ID NOS: 38 and 39. Examples of the primer combination for the two-stage PCR are exemplified by a combination of SEQ ID NOS: 18 and 19 for the first amplification and a combination of SEQ ID NOS: 19 and 20 for the second amplification.

As described in Examples, it has been clarified that the mutation occurs extremely scarcely in the nucleic acid of the present invention, wherein the sequence conservation is extremely high. Therefore, it is possible for those skilled in the art to easily understand that primer combinations, which have the equivalent performance, exist other than the primer combinations specifically disclosed in this specification. The primer combinations as described above are also included in the scope of the present invention.

The present invention also provides an expression vector in which the nucleic acid of the present invention or a part thereof is incorporated. In this specification, the term “expression vector” means the vector which inserts an exogenous polynucleotide into a host cell gene in an expressible state. Specifically, the expression vector is the vector which has a control sequence capable of incorporating the polynucleotide. The term “part” means the portion which encodes a peptide sufficient to allow a gene product to function or sufficient to exhibit the antigenicity.

The expression vector can be effectively used to obtain a virus peptide having the immunological activity or the biological activity. At present, various vectors are known as the vector for incorporating a gene desired to be expressed. Further, various host cells are also known as the host cell into which the gene is incorporated by using the vector to finally express the peptide.

The expression vector of the present invention is usually a recombinant expression vector which has the nucleotide sequence of the nucleic acid of the present invention or ORF thereof, wherein the ORF is connected to be operable with respect to the control sequence compatible to a desired host in the recombinant expression vector. A recombinant gene expression system can be constructed by using the expression vector.

In this specification, the term “transformed cell” means the cell capable of expressing all or a part of the polypeptide encoded by the nucleic acid of the present invention or a part thereof.

The transformed cell of the present invention can be obtained by transforming a host cell by directly introducing the polynucleotide including all or a part of the nucleic acid of the present invention or by introducing the recombinant expression vector incorporated with the polynucleotide into the host cell. In the present invention, it is possible to use a known transfer vector and a host cell.

The polypeptide, which is encoded by the nucleic acid of the present invention, can be produced by culturing the host cell as described above and recovering the expressed polypeptide from a culture. The culture condition for the host cell is appropriately selected depending on the host cell to be used. The condition for recovering the polypeptide is appropriately selected depending on the property of the object polypeptide.

When the gene product encoded by the novel gene or the fragment thereof can be once obtained, the gene product or the fragment thereof can be used as the antigen to detect and/or separate the antibody against the gene product or the fragment thereof existing in a biological sample. The method for detecting and/or separating the antibody against the antigen, which is based on the use of the antigen, is well-known in the concerning technical field.

The gene product or the fragment thereof can be also used to prepare and/or purify the antibody against the gene product or the fragment thereof. Various methods are available to prepare or purify the antibody by using the polypeptide including the gene product or the fragment thereof as the immune antigen or the probe, which are well-known in the concerning technical field.

When the fragment of the gene product is used as the immune antigen, it is desired that the fragment contains the epitope of the gene product. A large number of methods are disclosed to specify the position of the epitope existing in the polypeptide. It is also possible to seek out an epitope candidate from the elucidated amino acid sequence by using a commercially available software. The technique for analyzing and determining the epitope is well-known in the concerning technical field, and any analysis tool is commercially supplied.

The present invention will be explained more specifically below in accordance with Examples. However, the present invention is not limited to Examples without deviating from the gist or essential characteristics thereof. Unless otherwise specified, Examples shown below were carried out in accordance with the standard method (see, for example, Sambrook et al., MOLECULAR CLONING, A LABORATORY MANUAL, 2nd Ed; Cold Spring Harbor Laboratory Press, Cold Spring Harbor, N.Y. (1989)).

### Example 1

### <1> Setting and Preparation of Primers for Detecting Helicase Gene

It was speculated that any unknown hepatitis pathogen highly possibly existed in blood which provided a negative result for the screening test for the B type hepatitis virus and the C type hepatitis virus as known blood infectious hepatitis viruses although the value was abnormal in the ALT test which was the marker for indicating the liver function and which was used as one of the screening tests for transfusion bloods. Accordingly, the blood, which was abnormal in ALT and which was negative for the hepatitis virus marker, was selected as a starting material to obtain the unknown hepatitis pathogen.

A helicase family common primer was selected as a tool for seeking out the gene of the novel hepatitis pathogen candidate from the blood for the following reason, and the primer was prepared and used. It is known that the protein, which takes responsibility for the function more essential for the living or existence, exists commonly to many organisms, and the amino acid sequence thereof (and hence the nucleic acid sequence encoding the same) is conserved. Such a protein is also expected to exist in the objective novel hepatitis pathogen candidate gene. The following facts are known. That is, helicase is an enzyme having such an activity that the double strand structure of DNA/DNA or DNA/RNA or the secondary structure of RNA is converted into the single strand by cutting or cleaving the hydrogen bond. Helicase is one of the enzymes essential for the living or existence of the organism to widely participate, for example, in the replication of DNA, the repair, the recombination, the transcription of RNA, the splicing, and the translation into protein, and helicase exists in many organisms. In view of the above, the primer, which had the nucleic acid sequence common to the helicase family, was selected as the tool to be used to seek out the objective novel hepatitis pathogen gene. Sequence information was collected by screening the helicase family, and the sequence characteristics were investigated. Subsequently, the nucleic acid sequence of helicase of the virus was used as the basis, and the inosine insertion was performed therefor to prepare or manufacture helicase primers reactive with a wide range of helicase family genes.

That is, the primers for detecting the helicase gene to be used were prepared by performing the inosine insertion with respect to the sequence selected from the NS3 helicase consensus domain of the HCV helicase family. Specifically, the following three types of primers were prepared and used.

IA-3 (forward primer):
(SEQ ID NO: 40)
CCI ACI ggI AgI ggI AAR AgC AC;
IV-3 (reverse primer):
(SEQ ID NO: 41)
CTI CCM gTg CgI CCI CgS CgY Tg;
III R-2 (forward primer):
(SEQ ID NO: 42)
CCI ggR IIg TIg CIg TRg C.

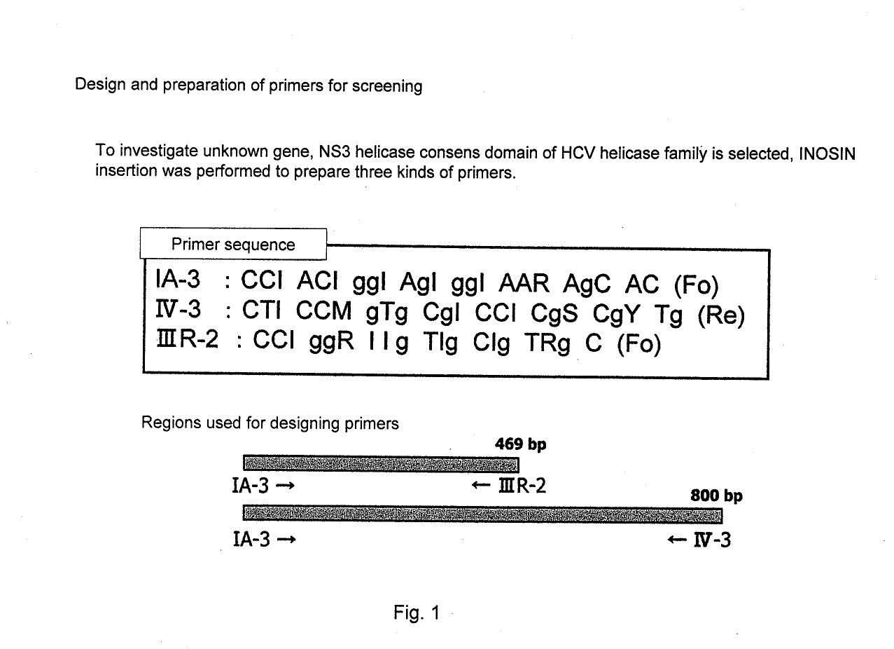

It was postulated that a gene fragment of 469 bp would be amplified from a combination of IA-3 and III R-2 and a gene fragment of 800 bp would be amplified from a combination of IA-3 and IV-3 (FIG. 1).

### <2> Amplification and Analysis of Unknown Pathogen Candidate Gene Fragment

It was tried to amplify the helicase gene (gene fragment of unknown pathogen candidate) for 500 samples of ALT abnormal human blood specimens by using the primers for detecting the helicase gene.

Nucleic acid was extracted from the specimen as follows by using ExR&D Smitest Kit (Medical & Biological Laboratories Co., Ltd.). 100 μl of blood plasma obtained from the specimen blood was introduced into a centrifuge tube to which 15 μL of Solution I (enzyme solution), 380 of Solution II (specimen diluent solution), and 5 μL of Solution IV (coprecipitation reagent solution) of the kit were added, followed by performing incubation at 55° C. for 30 minutes. Subsequently, 250 μL of Solution III (protein dissolvent solution) was added to perform incubation at 55° C. for 15 minutes, and then 600 μL of isopropanol was added to perform incubation in ice for 10 minutes. After the completion of incubation, the centrifugation was performed at 15,000 rpm for 10 minutes to obtain a precipitate while discarding a supernatant. 600 μL of 75% (V/v) ethanol was added thereto. The solution was centrifuged again at 15,000 rpm for 10 minutes, and an obtained precipitate was dried up. 30 μL of distilled water (DW) was added to the dried precipitate so that the precipitate was dissolved, and an obtained solution was provided as template DNA.

In order to amplify the helicase gene (unknown pathogen gene), the polymerase chain reaction (PCR), which was based on the use of TaKaRa Ex-Taq as an enzyme and which used the template DNA as a template, was performed by using TaKaRa PCR Thermal Cycler 480 amplifier. The PCR reaction solution contained the followings per tube: 20 μL of template DNA, 0.25 μL of Ex-Taq, 20 μL of 10× buffer, 1 μL of 10 mM dNTP, 0.5 μL of forward primer IA-3, 0.5 μL of reverse primer IV-3, and 22.75 μL of distilled water (total amount: 50 μL). The reaction condition of PCR was as follows. That is, the amplification cycle composed of the denaturation at 94° C. for 30 seconds, the annealing at 55° C. for 30 seconds, and the extension at 72° C. for 2 minutes was performed 35 times after the denaturation reaction at 94° C. for 2 minutes, and the extension was finally performed at 72° C. for 7 minutes.

The amplified gene was separated by means of the agarose gel electrophoresis (gel concentration: 1.5%, 20 mA, 30 minutes), the gene was thereafter stained with ethidium bromide for 30 minutes, and the gene was detected by being irradiated with the ultraviolet light. Twelve samples, in which the helicase gene (unknown pathogen candidate gene) was strongly amplified, were found out from 500 samples of the ALT abnormal human blood specimens.

Subsequently, the amplified nucleic acid was excised from the gel of the electrophoresis and purified by using MinElute Gel Extraction kit (Qiagen). Specifically, the weight of the gel was firstly measured. Buffer QG appended to the kit was added in an amount of 600 μL per 100 mg of the gel, followed by incubation at 50° C. for 10 minutes. After the completion of incubation, isopropanol, which was in a 1/10 amount (v/v) of the solution, was added thereto, which was added to MinElute Spin column to perform centrifugation at 15,000 rpm for 1 minute. Subsequently, 500 μL of Buffer QG was added to the column, followed by performing centrifugation at 15,000 rpm for 1 minute to wash impurities. Subsequently, 700 μL of Buffer PE was added, followed by performing centrifugation at 15,000 rpm for 1 minute to repeat the washing. Further, the centrifugation was performed at 15,000 rpm for 1 minute without adding any buffer to completely remove the washing solution from the column. After that, a tube was set under the column in order to receive the eluted matter. 10 μL of Buffer EB was added, followed by performing centrifugation at 15,000 rpm for 1 minute to elute the nucleic acid which was recovered in the tube disposed thereunder.

Subsequently, the sequence of the recovered amplified nucleic acid was determined in accordance with the following procedure. At first, the amplified nucleic acid was amplified by using Big Dye Terminator v1.1 cycle sequencing kit (ABI), GeneAmp PCR system 9700 (Applied Biosystems), and two types of (forward and reverse) amplification solutions having the following component compositions: forward (8 μL of Pre Mix, 4 μL of forward primer IA-3, 4 μL of template (nucleic acid), and 4 μL of distilled water) and reverse (8 μL of Pre Mix, 4 μL of reverse primer IV-3, 4 μL of template (nucleic acid), and 4 μL of distilled water). Subsequently, a reaction product (amplified nucleic acid) was purified by using DyeEx 2.0 Spin kit (Qiagen). Specifically, DyeEx 2.0 Spin column appended to the kit was centrifuged at 3,200 rpm for 3 minutes, and then the total amount of the reaction product was added to the column, followed by centrifugation at 3,200 rpm for 3 minutes to purify the reaction product. The purified amplified nucleic acid (liquid) was treated at 95° C. for 2 minutes. The sample was placed on ice (0° C.) for 10 minutes, followed by being applied to centrifugation, and then the total amount thereof was transferred to a sequence-measuring plate. The plate was analyzed by ABIPRISM 3100 Gene tic Analyzer by using Gene tic Analyzer with EDTA 3100 POP-6 polymer, and the sequence was determined. In the sequence determination, DT3100POP6 (BD) v2.mob was used as Mobility File, SR-Seq50-POPE-20S-5400sec was used as Run module, and BC-3100SR-SeqOffFtOff was used as Analysis module. The obtained data was analyzed by using an analysis software SEQUENCER GENETYX-MAC.

The homology screening was performed by using DDBJ BLAST and NCBI BLAST. As for the sequence, the homology screening was firstly performed. The open reading frame analysis, the translation into the amino acid sequence, the translation into the amino acid sequence after performing the complementary conversion, the homology screening for the amino acid sequence, the restriction enzyme site analysis, and the GC content distribution analysis were carried out for those having any possibility of the unknown pathogen candidate gene. As a result, it was revealed that the amplified sequences were sequences of bacteria and G type hepatitis virus (i.e., known pathogen sequences) for 8 samples of 12 sample which exhibited the strong positive result in the amplification based on the use of the helicase common primer. Remaining 4 samples exhibited the same sequence, for which it was revealed that the homology screening score (bits) was not more than 50, the E value was 3.5, and the sequence had a high possibility of novel sequence. Therefore, it was intended to clarify the entire sequence by subsequently performing the extension of the gene.

### <3> Determination of Sequence of Unknown Pathogen Candidate Gene

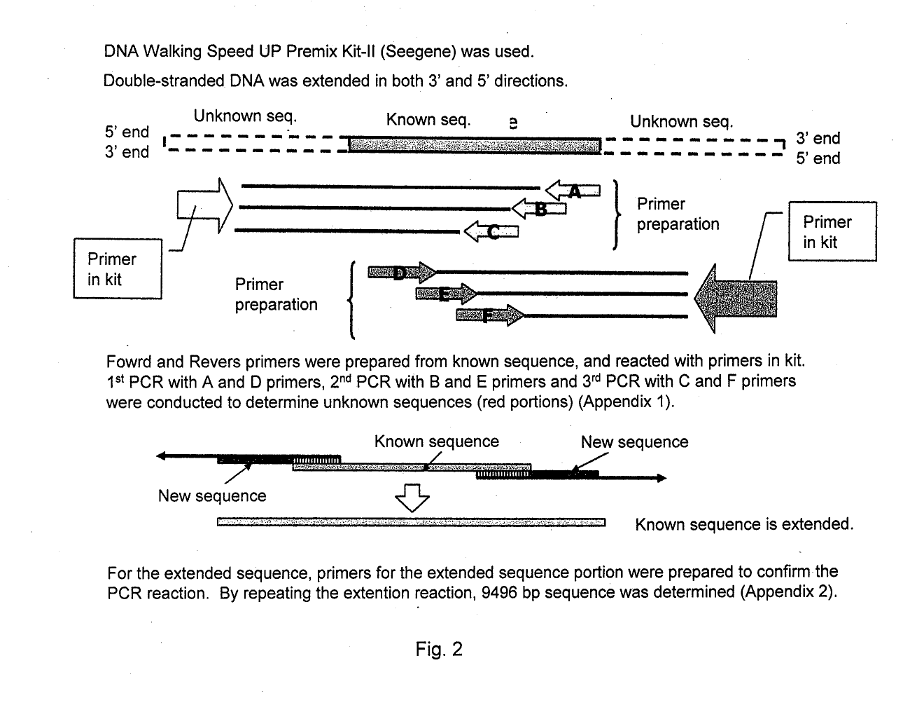

FIG. 2 shows an outline of the primer walking method by which the gene sequence of the present invention was determined. A plurality of forward primers (A, B, C) and reverse primers (D, E, F) were manufactured on the basis of the gene sequence of the unknown pathogen candidate determined in accordance with <2>. The primers were used in accordance with an explanatory pamphlet for the handling appended to the kit together with DNA Walking Speed UP Premix Kit-II (Seegene) to determine the undetermined gene sequence of the unknown pathogen candidate (indicated as unknown Seq in FIG. 2). Specifically, Primer 1 and Primer A appended to the kit were used to carry out the nucleic acid (DNA) amplification by using, as the template, the nucleic acid fraction in which the unknown pathogen candidate gene fragment was amplified (1st PCR). Subsequently, DNA amplified in 1st PCR was used as the template to perform the 2nd DNA amplification (2nd PCR) by using Primer 2 and Primer B appended to the kit. Further, DNA amplified in 2nd PCR was used as the template to perform the 3rd DNA amplification (3rd PCR) by using Primer 3 and Primer C appended to the kit. DNA's obtained in the respective amplification processes were gel-purified to determine the sequences of the amplified nucleotides amplified by the sequencing operation as described above. This operation was repeatedly carried out until any new sequence was not determined to determine the entire nucleic acid sequence of the unknown pathogen candidate gene (SEQ ID NO: 1, hereinafter referred to as “nucleic acid sequence of the invention”). The compositions of the reaction solutions and the reaction conditions of 1st PCR, 2nd PCR, and 3rd PCR were as follows.

TABLE 1
1st PCR
Four tubes were prepared for one specimen.

| Composition of PCR solution: | | |
|---|---|---|
| Template DNA: | 5 | μL |
| DW2-ACP (1-4): | 2 | μL |
| 1 or A: | 1 | μL |
| Distilled water: | 2 | μL |
| 2 x seeAmp Acp MasterMix: | 10 | μL |
| total | 20 | μL |
| PCR condition: | | |
| 94° C.: | 5 | minutes |
| 42° C.: | 1 | minute |
| 72° C.: | 2 | minutes |
| Cycle of 94° C. (0.5 minute)-60° C. (0.5 minute)-72° C. | 30 | times |
| (100 seconds): | | |
| 72° C.: | 7 | minutes |

2nd PCR

| | | |
|---|---|---|
| Composition of PCR solution: | | |
| 1st PCR product: | 4 | μL |
| DW2-ACP (1-4): | 2 | μL |
| 2 or B: | 1 | μL |
| Distilled water: | 2 | μL |
| MasterMix: | 10 | μL |
| total | 20 | μL |
| PCR condition: | | |
| 94° C.: | 3 | minutes |
| Cycle of 94° C. (0.5 minute)-60° C. (0.5 minute)-72° C. | 30 | times |
| (1 minute): | | |
| 72° C.: | 7 | minutes |

3rd PCR

| | | |
|---|---|---|
| Composition of PCR solution: | | |
| 50-fold solution of 2nd PCR product: | 5 | μL |
| Uni P2: | 1 | μL |
| 3 or C: | 1 | μL |
| Distilled water: | 3 | μL |
| 2 x seeAmp Acp MasterMix: | 10 | μL |
| total | 20 | μL |
| PCR condition: | | |
| 94° C.: | 3 | minutes |
| Cycle of 94° C. (0.5 minute)-60° C. (0.5 minute)-72° C. | 30 | times |
| (1 minute): | | |
| 72° C.: | 7 | minutes |

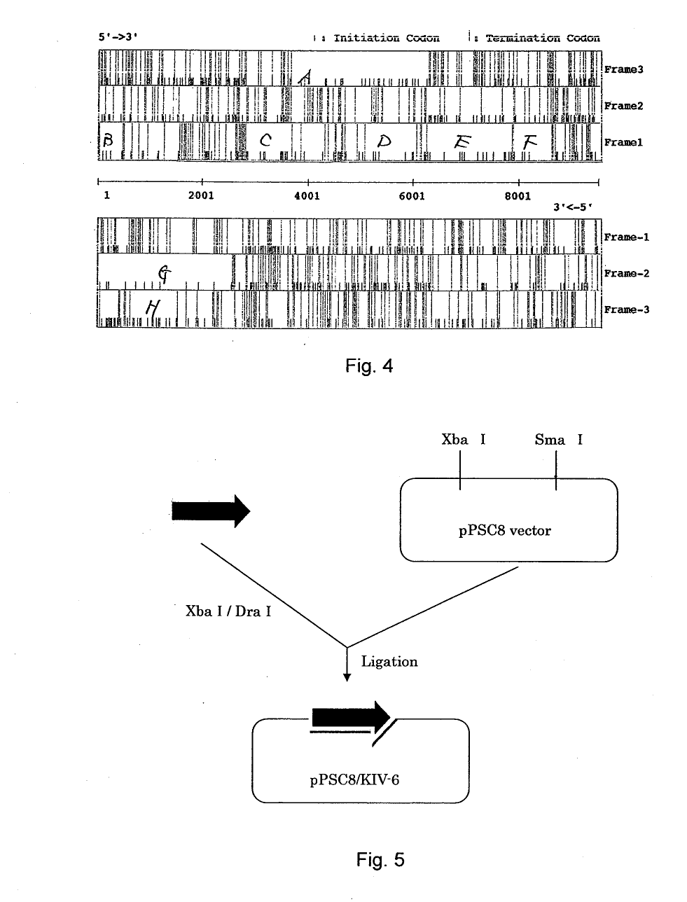

The nucleotide sequence determined as described above (nucleic acid sequence of the present invention) is shown in SEQ ID NO: 1. FIG. 4 shows a result of the screening for the open reading frame candidate on the basis of the nucleotide sequence. As a result, 8 open reading frame candidates were found out. The nucleotide sequences thereof are shown in SEQ ID NOS: 2 to 9, and the amino acid sequences encoded by the nucleotide sequences are shown in SEQ ID NOS: 10 to 17.

### Example 2

### Method for Detecting Nucleic Acid Sequence of the Present Invention

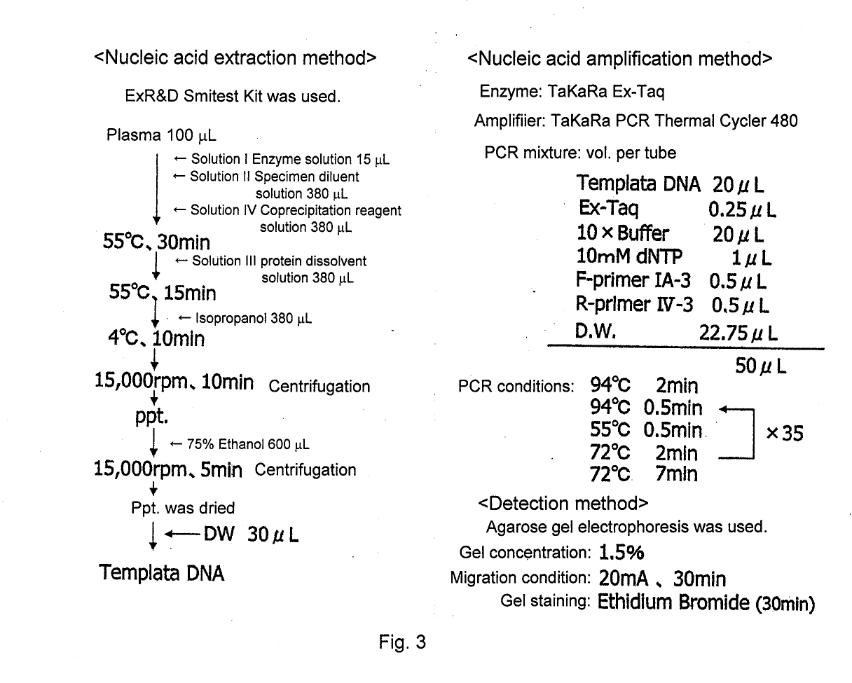

A plurality of polynucleotides were prepared on the basis of the nucleic acid sequence clarified in Example 1 to investigate whether or not the polynucleotides were usable as the primer. Specifically, oligonucleotides of SEQ ID NOS: 18 to 39 were synthesized. The ability to amplify the nucleic acid sequence of the invention was tested for combinations of SEQ ID NOS: 18 and 19, 19 and 20, 22 and 23, 24 and 25, 26 and 27, 28 and 29, 30 and 31, 32 and 33, 34 and 35, 36 and 37, and 38 and 39. It was confirmed that they were usable equivalently. The test condition was as shown in FIG. 3. Therefore, it was confirmed that the nucleic acid sequence of the present invention was successfully detected by means of PCR based on the use of the primers.

### Example 3

### Presence of Nucleic Acid Sequence of the Present Invention in Clinical Specimen Including Patient of Hepatitis of Unknown Cause

If the nucleic acid sequence of the present invention originated from the pathogen of the hepatitis of unknown cause, it was expected that the nucleic acid sequence of the present invention would not be detected in normal human and the nucleic acid sequence of the present invention would be detected in a case of disease of hepatitis of unknown cause. In order to verify the clinical usefulness of the nucleic acid sequence of the invention, it was investigated whether or not the nucleic acid sequence of the present invention was present, by using the nucleic acid sequence detecting method established in Example 2 (primers to be used: SEQ ID NOS: 18 and 19 (1st PCR) and SEQ ID NOS: 20 and 21 (2nd PCR) in relation to specimens of normal human, non-A, non-B, and non-C hepatitis patients, specimens of patients of known viral hepatitis (HBV and HCV), and specimens (plasmas) of patients of liver diseases, i.e., fatty liver, cholestatic hepatitis, primary biliary hepatitis, alcoholic hepatitis, and alcoholic cirrhosis. Obtained results are shown in the following table.

TABLE 2
Detection rate of nucleic acid sequence of the invention in
patients of non-A, non-B, and non-C hepatitises and
patients of various liver diseases

| | Positive number/ | Positive |
|---|---|---|
| Patient group | total test number | rate |
| Normal human | 0/14 | 0% |
| non-A, non-B, non-C hepatitis | 6/12 | 50% |
| patients | | |
| Known viral hepatitis patients | 5/56 | 9% |
| Fatty liver | 1/7 | 15% |
| Cholestatic hepatitis | 1/4 | 25% |
| Primary biliary hepatitis | 14/24 | 58% |
| Alcoholic hepatitis | 1/11 | 9% |
| Alcoholic cirrhosis | 7/10 | 70% |

As shown in the results, the positive rate was extremely low in the normal human and the known viral hepatitis patients. On the other hand, the positive rate was extremely high in the non-A, non-B, and non-C hepatitis patients, the primary biliary hepatitis, and the alcoholic cirrhosis. The causes of the diseases of the primary biliary hepatitis and the alcoholic cirrhosis have not been decided yet. A possibility is suggested such that the pathogen, which has the novel nucleic acid of the present invention, may be the cause.

### Example 4

### Properties of Nucleic Acid Sequence of the Present Invention

### (1) Possibility or Impossibility of Reverse Transcription

ExR&D Smitest Kit (Medical & Biological Laboratories Co., Ltd.) was used to extract the nucleic acid from the specimen. 15 μL of Solution I (enzyme solution), 380 μL of Solution II (specimen diluent solution), and 5 μL of Solution IV (coprecipitation reagent solution) of the kit were added to 100 μL of blood plasma, followed by performing incubation at 55° C. for 30 minutes. Subsequently, 250 μL of Solution III (protein dissolvent solution) was added to perform incubation at 55° C. for 15 minutes, and then 600 μL of isopropanol was added to perform incubation in ice for 10 minutes. After the completion of incubation, the centrifugation was performed at 15,000 rpm for 10 minutes to obtain a precipitate to which 600 μL of 75% ethanol was added. The centrifugation was performed again at 15,000 rpm for 10 minutes, and an obtained precipitate was dried up. 30 μL of distilled water was added to the dried precipitate so that the precipitate was dissolved. After extracting the nucleic acid, DNase of a final concentration of 10 U and magnesium of a final concentration of 10 mM were added to perform a treatment at 37° C. for 30 minutes. 50 μL of a mixture solution of phenol:chloroform (1:1) was added to the treatment solution, followed by being agitated and centrifuged at 15,000 rpm for 15 minutes. 1/10 volume of 8 M ammonium acetate and 2.5-fold volume of ethanol were added to a supernatant, followed by being stationarily placed at −80° C. for 20 minutes. The centrifugation was performed at 15,000 rpm for 20 minutes, and an obtained precipitate was recovered as the nucleic acid. 20 μL of distilled water was added to the obtained nucleic acid. 2 μL of 0.1 M DTT, 1 μL of 10 mM dNTP, 1 μL of 40 U/μL of RNase (ribonuclease) inhibitor (Rnasin, PROMEGA), 1 μL of 200 U/μL of reverse transcriptase were added to 10 μL of the nucleic acid solution, followed by being reacted at 42° C. for 50 minutes to synthesize cDNA. After further performing the reaction of 1st PCR, an objective amplification product was detected by means of the gel electrophoresis. As a result, the amplification product was not detected. Therefore, it was judged that the objective nucleic acid was not RNA but DNA.

Further, after extracting the nucleic acid, RNase treatment was performed. 50 μL of a mixture solution of phenol:chloroform (1:1) was added to a reaction solution, followed by being agitated. The centrifugation was performed at 15,000 rpm for 15 minutes. 1/10 volume of 8 M ammonium acetate and 2.5-fold volume of ethanol were added to an obtained supernatant, followed by being stationarily placed at −80° for 20 minutes. The centrifugation was performed at 15,000 rpm for 20 minutes, and a precipitate was recovered as the nucleic acid. The reaction of 1st PCR was performed by using the obtained nucleic acid. As a result, the amplification product was detected. According to the results as described above, it was revealed that the objective nucleic acid was DNA.

(2) Demonstration of state of Nucleic Acid (to Verify Free State or State of Being Surrounded by Capsid

4 μL of Cloned DNase 1 (TaKaRa) of a final concentration of 8 U and magnesium of a final concentration of 10 mM were added to 100 μL blood plasma, followed by being treated at 37° C. for 30 minutes. A control was provided, which was prepared by treating extracted DNA with DNase 1, without providing blood plasma. The nucleic acid was extracted from the specimen after the treatment by using ExR&D Smitest Kit (Medical & Biological Laboratories Co., Ltd.) as follows. 15 μL of Solution I (enzyme solution), 380 μL of Solution II (specimen diluent solution), and 5 μL of Solution IV (coprecipitation reagent solution) of the kit were added to 100 μL of blood plasma, followed by performing incubation at 55° C. for 30 minutes. Subsequently, 250 μL of Solution III (protein dissolvent solution) was added to perform incubation at 55° C. for 15 minutes, and then 600 μL of isopropanol was added to perform incubation at 4° C. for 10 minutes. After the completion of incubation, the centrifugation was performed at 15,000 rpm for 10 minutes to obtain a precipitate to which 600 μL of 75% ethanol was added. The centrifugation was performed again at 15,000 rpm for 10 minutes, and an obtained precipitate was dried up. 30 μL of distilled water was added to the dried precipitate so that the precipitate was dissolved. A 10-fold dilution series of the dissolved nucleic acid was prepared to measure the end titer. Primers of SEQ ID NOS: 18 and 19 were used under the condition of 1st PCR, and SEQ ID NOS: 20 and 21 were used for 2nd PCR. As a result, the specimen not treated with DNase and the specimen treated with DNase were detected while providing the same value of the end titer. The amplification was not detected for a specimen in which the extracted DNA was treated with DNase in order to measure the function and effect of DNase. This resulted from the fact that DNase functioned sufficiently. According to the results as described above, it was successfully demonstrated that DNA did not exist in the free state in blood plasma but DNA existed in a state of being surrounded by capsid.

### (3) Restriction Enzyme Reaction SalI Digestion

ExR&D Smitest Kit (Medical & Biological Laboratories Co., Ltd.) was used to extract the nucleic acid from the specimen. 15 μL of Solution I (enzyme solution), 380 μL of Solution II (specimen diluent solution), and 5 μL of Solution IV (coprecipitation reagent solution) of the kit were added to 100 μL of blood plasma, followed by performing incubation at 55° C. for 30 minutes. Subsequently, 250 μL of Solution III (protein dissolvent solution) was added to perform incubation at 55° C. for 15 minutes, and then 600 μL of isopropanol was added to perform incubation in ice for 10 minutes. After the completion of incubation, the centrifugation was performed at 15,000 rpm for 10 minutes to obtain a precipitate to which 600 μL of 75% ethanol was added. The centrifugation was performed therefor again at 15,000 rpm for 10 minutes, and an obtained precipitate was dried up. 45 μL of distilled water was added to the dried precipitate so that the precipitate was dissolved. After the dissolution, 5 μL of 10-fold concentration H buffer (buffer appended to restriction enzyme) (produced by TaKaRa) and 4 μL of SalI (produced by TaKaRa) (20,000 U/ml) were added, followed by being mixed and reacted at 37° C. for 90 minutes. 50 μL of phenol:chloroform (1:1) mixture solution was added to the reaction solution, followed by being agitated. The centrifugation was performed at 15,000 rpm for 15 minutes. 1/10 volume of 8 M ammonium acetate and 2.5-fold volume of ethanol were added to an obtained supernatant, followed by being stationarily placed at −80° C. for 20 minutes. The centrifugation was performed at 15,000 rpm for 20 minutes to recover the nucleic acid as a precipitate. DNA not subjected to the Sail enzyme treatment was provided as a control to perform the investigation. PCR was performed with primers interposing the restriction enzyme cleavage site. As a result, the amplification was not detected for DNA digested with SalI, but the amplification was detected for DNA not digested with SalI. According to this fact, it was successfully demonstrated that the objective DNA is cut by the restriction enzyme and the DNA is double strand DNA.

### Example 5

### Screening for Nucleotide Sequence in Clinical Specimen

The screening for the nucleotide sequence was performed in relation to the clinical specimen which was PCR positive in Example 2. Long-PCR (LA-taq) was firstly performed for the extracted nucleic acid. The composition of the reaction solution included 0.5 μL of LA-taq, 25 μL of 2×GC(1) buffer, 8 μL of 2.5 mM dNTP, 0.5 μL of the primer of SEQ ID NO: 22, 0.5 μL of the primer of SEQ ID NO: 39, and 16 μL of the extracted nucleic acid solution. PCR was performed under the following PCR condition.

TABLE 3

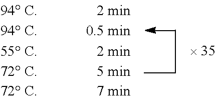

After the completion of LA-Taq PCR, PCR was performed again with the following combinations of the primers including the nucleotide sequences of SEQ ID NOS: 22 to 39 or the complementary sequences thereof. The condition shown in FIG. 3 was adopted as the condition to perform PCR. After the completion of PCR, the agarose gel electrophoresis was performed, the band was cut out, and the sequencing reaction was performed to determine the nucleotide sequence. Sequence coincidence of 98 to 100% was confirmed by making comparison with the nucleotide sequence of SEQ ID NO: 1 already determined.

TABLE 4

| | |
|---|---|
| Primer | |
| combination | SEQ ID NOS: |

| | |
|---|---|
| 1. | 22-23 |
| 2. | 23-24 |
| 3. | 24-25 |
| 4. | 25-26 |
| 5. | 26-27 |
| 6. | 27-28 |
| 7. | 29-30 |
| 8. | 30-31 |
| 9. | 31-32 |
| 10. | 32-33 |
| 11. | 33-34 |
| 12. | 34-35 |
| 13. | 35-36 |
| 14. | 36-37 |
| 15. | 37-38 |
| 16. | 38-39 |

### Example 6

### Expression of Protein Encoded by Open Reading Frame (ORF)

### (1) Construction of Expression Vector

Target genes, which contained five ORF's (ORF's of No. 1: 3070 to 3669 bp, No. 2: 3826 to 4293 bp, No. 3: 5233 to 6117 bp, No. 4: 7198 to 7872 bp, No. 5: 7888 to 8610 bp) included in ORF's existing in the nucleotide sequence of SEQ ID NO: 1, were amplified by using primers shown in the following table, and 6×His PCR products having XbaI, DraI restriction enzyme sites were obtained.

TABLE 5
Expression primers
<Forward>

| | | | | SEQ |
|---|---|---|---|---|
| | Primer | | | ID |
| ORF | name | F common (30mer) | Target | NO |
| 1-1 | 1-1F | TGATCTAGAATGCATCATCATCATCATCAT- | TCTCGTGAACTCGTCTCGTCGTCATC | 43 |
| 1-2 | 1-2F | TGATCTAGAATGCATCATCATCATCATCAT- | GAATGGCATCTCCAACCCAAGATATC | 44 |
| 1-3 | 1-3F | TGATCTAGAATGCATCATCATCATCATCAT- | AAGAGGAAGTACAGGATCCATGTAGTG | 45 |
| 1-4 | 1-4F | TGATCTAGAATGCATCATCATCATCATCAT- | CGATCTGTGGTCGAGGTTTCATCTC | 46 |
| 1-5 | 1-5F | TGATCTAGAATGCATCATCATCATCATCAT- | AATAGGGAAATGATCTATACCCGCGT | 47 |
| | | Xba I HisHisHisHisHisHis | | |

<Reverse>

| | | | | SEQ |
|---|---|---|---|---|
| | Primer | | | ID |
| ORF | name | R common | Target origin | NO |
| 1-1 | 1-1R | TCATTTAAA- | TCAGAGTATGCTGCTGAGAGTTG | 48 |
| 1-2 | 1-2R | TCATTTAAA- | TCAATTCGGCTGGAGTGACTG | 49 |
| 1-3 | 1-3R | TCATTTAAA- | CTATTCGGAGGGGGGATGCGAT | 50 |
| 1-4 | 1-4R | TCATTTAAA- | TCACGGAAGATGGACGTACCTTG | 51 |
| 1-5 | 1-5R | TCATTTAAA- | CTAAAGCGGTCTACTCTGAAC | 52 |
| | | Dra I | | |

The 6×His PCR product obtained as described above was subjected to the restriction enzyme treatment with XbaI/DraI. Subsequently, it was confirmed that the fragment having the objective length was obtained by means of the agarose gel electrophoresis. The fragment was mixed with pPSC8 (Nosan Corporation) enzyme-treated with XbaI/SmaI to perform the ligation reaction (FIG. 5). E. coli DH5α was transformed with the ligation product, and a clone, which had the objective sequence, was selected from transformants (pPSC8/KIV-6). Obtained sequences were analyzed, and the identical sequence was confirmed.

### (2) Cotransfection

200 μL of Sf900II (Invitrogen) including about 2 μg of expression vector DNA, 85 ng of Linear AcNPV DNA (Nosan Corporation), and 5 μL of Insect GeneJuice (Merck) was added to Sf9 cells (Nosan Corporation) of 1.0×10⁶ cells seeded in a 25 cm² flask. The cells were cultured at 28° C. for about 6 days, and then a culture supernatant was recovered (cotransfection solution).

### (3) Simplified Production of Expression Product

SF+ cells (Nosan Corporation), which were diluted with Sf900II to provide 1.5×10⁶ cells/mL, were prepared in an amount of 100 mL in a 250 mL Erlenmeyer flask. The cotransfection solution prepared in (2) was added thereto, and the cells were cultured with shaking at 130 rpm at 28° C. The cell culture solution was recovered after 72 hours, and the centrifugation was performed at 3,000×g at 4° C. for 30 minutes to perform the fractionation into a precipitate and a supernatant.

### (4) Purification of 6×His Binding Expression Protein

The precipitate and the supernatant, which were centrifuged and separated at 3,000×g, were purified in accordance with the His Trap method by using His Trap Affinity Columns produced by NoVagen.

### Example 7

### Detection of Antibody Against Expressed Polypeptide

Any antibody contained in the specimen was detected by means of EIA based on the use of a solid phase on which the protein purified in Example 6 was immobilized. The followings were used as the specimen (blood plasma). General specimen: normal human specimen having ALT value of not more than 61 IU/L;

ALT abnormal human specimen: specimen suffered from hepatopathy having ALT value of not less than 61 IU/L.

EIA was carried out as follows.

### EIA (Measurement of Antibody)

Method: The sample was diluted in 0.2 M Carbonate Buffer pH 9.5 so that the protein amount was 5 μg/well, followed by being left to stand in 96-well plate at 4° C. overnight to form a solid phase. After forming the solid phase (immobilization), the washing was performed three times with a washing solution (23.8 g of Na₂HPO₄.12H₂O, 5.23 g of NaH₂PO₄.2H₂O, 42.5 g of NaCl, 2.5 ml of Tween 20/L). 100 μL of 20% bovine serum/PBS was added to perform the blocking for 60 minutes. After the blocking, the washing was performed three times, and 50 μL of the specimen, which was diluted 50 times with 20% bovine serum/PBS, was added to perform the reaction for 60 minutes. After the reaction, the washing was performed five times, and anti-Human IgG Peroxidase labeled antibody (Jackson Immuno Research), which was diluted 5000 times with 20% bovine serum/PBS, was added to perform the reaction for 60 minutes. After the reaction, the washing was performed five times. The color was developed with OPD (for 30 minutes in a dark place), and then the measurement was performed at two wavelengths of 492 nm and 640 nm.

The suppression test was performed with the expressed protein in relation to the specimen which exhibited the positive result. The suppression test was performed in the same manner as the method for measuring the specimen except that a specimen to be used was obtained by adding 50 μg of the expressed protein to the EIA positive specimen and performing the reaction for 60 minutes. Those having the suppression rate of not less than 50% were designated as the antibody positive.

The presence or absence of the nucleic acid sequence was also measured for the specimen described above by using the nucleic acid sequence detecting method described in Example 2. The primers used were as follows, and the condition of PCR was as shown in FIG. 3.

1st PCR: SEQ ID NO: 18 primer 101-C
: SEQ ID NO: 19 primer N101-B

2nd PCR: SEQ ID NO: 20 primer KS-2
SEQ ID NO: 21 primer N101-D

Results are shown in the following table. In the table, PCR− indicates the PCR negative, and PCR+ indicates the PCR positive. According to the results, it is acknowledged that the antibody against the expressed protein exists in the ALT abnormal specimen.

TABLE 6
Results:

| Objective ORF | | No. 1 | No. 2 | No. 3 | No. 4 | No. 5 |
|---|---|---|---|---|---|---|
| General specimen | PCR− | 0/100 | 0/100 | 0/100 | 0/100 | 0/100 |
| ALT abnormal | PCR− | 18/100 | 10/100 | 20/100 | 22/100 | 5/100 |
| specimen | PCR+ | 0/6 | 0/6 | 0/6 | 0/6 | 0/6 |

### Example 8

### Immunoprecipitation Reaction of Virus with EIA Positive Specimen

The immunoprecipitation reaction of virus was investigated by means of the following method by using three samples of positive specimens and three samples of negative specimens which exhibited the positive and negative results with respect to the expressed proteins Nos. 1, 2, 3, and 4 in Example 6 respectively.

100 μL of the PCR positive specimen and 100 μL of the EIA positive specimen or the EIA negative specimen diluted 50 times were mixed with each other, followed by being reacted for 60 minutes (each of the specimens was allowed to pass through a 0.2 μm filter as a pretreatment before the mixing and the centrifugation was performed at 15,000 rpm for 10 minutes). After the reaction, the centrifugation was performed at 12,000 rpm for 10 minutes. A supernatant was discarded and a pellet was washed three times with Saline (centrifugation at 12,000 rpm for 5 minutes). After the washing, 100 μL of Saline was added, and the nucleic acid was extracted to perform the PCR measurement.

Results are shown in the following table. According to the results, it is acknowledged that the antibody contained in the EIA positive specimen recognizes the virus particles.

TABLE 7
Results of PCR measurement of respective immunoprecipitates

| | PCR | | |
|---|---|---|---|
| Only PCR positive specimen | − | | |
| PCR positive specimen and Saline | − | | |
| PCR positive specimen and EIA positive specimen | + | + | + |
| PCR positive specimen and EIA negative specimen | − | − | − |

### DESCRIPTION OF SEQUENCES

SEQ ID NO: 1: nucleotide sequence of KIV9496 (9496 nucleotides)
SEQ ID NO: 2: nucleotide sequence of deduced ORF-A
SEQ ID NO: 3: nucleotide sequence of deduced ORF-B
SEQ ID NO: 4: nucleotide sequence of deduced ORF-C
SEQ ID NO: 5: nucleotide sequence of deduced ORF-D
SEQ ID NO: 6: nucleotide sequence of deduced ORF-E
SEQ ID NO: 7: nucleotide sequence of deduced ORF-F
SEQ ID NO: 8: nucleotide sequence of deduced ORF-G
SEQ ID NO: 9: nucleotide sequence of deduced ORF-H
SEQ ID NO: 10: amino acid sequence of deduced ORF-A
SEQ ID NO: 11: amino acid sequence of deduced ORF-B
SEQ ID NO: 12: amino acid sequence of deduced ORF-C
SEQ ID NO: 13: amino acid sequence of deduced ORF-D
SEQ ID NO: 14: amino acid sequence of deduced ORF-E
SEQ ID NO: 15: amino acid sequence of deduced ORF-F
SEQ ID NO: 16: amino acid sequence of deduced ORF-G
SEQ ID NO: 17: amino acid sequence of deduced ORF-H

SEQ ID NO: 18:
primer 101-C CAACACCgCAATCACAAAgT (3007-3026)
SEQ ID NO: 19:
primer N101-B AACATTgAAACgTCATgTCC (3445-3464)
SEQ ID NO: 20:
primer KS-2 CTCgTCTCgTCgTCATCgTA (3082-3101)
SEQ ID NO: 21:
primer N101-D CATTTgCTCCCgCTggAgATg (3365-3385)
SEQ ID NO: 22:
primer K-5 AACgATCCggACATCCACCACCA (145-167)
SEQ ID NO: 23:
primer KIV-21 gCgATCgAggCATggCTCAA (536-555)
SEQ ID NO: 24:
primer 101-la CTgCAgAgCATCATgCggAg (796-815)
SEQ ID NO: 25:
primer 101-T TCgATCCgCTTTCggTACgT (1383-1402)
SEQ ID NO: 26:
primer X-2 ACCTAATgAAgACgCCgAAT (2290-2309)
SEQ ID NO: 27:
primer KS-1 gAgACATggTgTAAgAgTCg (3057-3076)
SEQ ID NO: 28:
primer 101-11 AgTggATgAAgCTggACCTT (3887-3906)
SEQ ID NO: 29:
primer Op-3 gATCTTgATCCTgTACTCTT (4416-4435)
SEQ ID NO: 30:
primer 101-21 gCgAAAgAggATTCTCgACT (4788-4807)
SEQ ID NO: 31:
primer X-7 TgggAgTATggAgTCgACAT (5331-5350)
SEQ ID NO: 32:
primer 101-23 TACACTAAAATCgACTCCTCC (6047-6067)
SEQ ID NO: 33:
primer X-13 ATgAgTTTgAgAACgATCTT (6733-6752)
SEQ ID NO: 34:
primer 101-29 ACCTgCgCCTgAggCTACgA (6888-6907)
SEQ ID NO: 35:
primer X-17 gCAgATCgTTTCCgTTTggg (7564-7583)
SEQ ID NO: 36:
primer 101-38 AACATCgAAAgATTAAAgAAA (7940-7960)
SEQ ID NO: 37:
primer 101-6R CACgCgATTCCCATATCCCT (8789-8808)
SEQ ID NO: 38:
primer KIV-14 CgTgTACTAACTATACTgAC (9255-9274)
SEQ ID NO: 39:
primer KIV-16 CTCAAATTCCATCCgAATAg (9425-9444)

SEQ ID NO: 40: helicase gene detecting primer IA-3
SEQ ID NO: 41: helicase gene detecting primer IV-4
SEQ ID NO: 42: helicase gene detecting primer III R-2
SEQ ID NO: 43: primer 1-1F
SEQ ID NO: 44: primer 1-2F
SEQ ID NO: 45: primer 1-3F
SEQ ID NO: 46: primer 1-4F
SEQ ID NO: 47: primer 1-5F
SEQ ID NO: 48: primer 1-1R
SEQ ID NO: 49: primer 1-2R
SEQ ID NO: 50: primer 1-3R
SEQ ID NO: 51: primer 1-4R
SEQ ID NO: 52: primer 1-5R

1. A nucleic acid comprising a nucleotide sequence of SEQ ID NO: 1 or a nucleotide sequence complementary to a nucleotide sequence of SEQ ID NO: 1.

2. (canceled)

3. A nucleic acid comprising a nucleotide sequence of an open reading frame encoding a protein having a length of not less than 20 amino acids in a nucleotide sequence of SEQ ID NO: 1.

4. A polypeptide comprising an amino acid sequence encoded by the open reading frame of the nucleic acid as defined in claim 3 or an amino acid sequence of a fragment thereof having a length of not less than 20 amino acids.

5. A polynucleotide which has a nucleotide length of not less than 10 bases, which includes a partial nucleotide sequence of a nucleotide sequence of SEQ ID NO: 1 or a nucleotide sequence complementary to the nucleotide sequence, and which is specifically hybridizable to the nucleic acid as defined in claim 1.

6. The polynucleotide according to claim 5, wherein the polynucleotide has a nucleotide length of not more than 50 bases.

7. The polynucleotide according to claim 5, wherein the polynucleotide includes any one of nucleotide sequences of SEQ ID NOS: 18 to 38.

8. A method for detecting a nucleic acid including a nucleotide sequence of SEQ ID NO: 1, a sequence complementary thereto, or a partial sequence of any one thereof, the method comprising using one or more of the polynucleotides as defined in any one of claims 5 to 7, as a primer or primers and/or a probe or probes.

9. The method according to claim 8, further comprising amplifying the nucleic acid by using the polynucleotide or polynucleotides as the primer or primers.

10. The method according to claim 9, wherein a combination of the polynucleotides to be used as the primers is any one of combinations of SEQ ID NOS: 18 and 19, SEQ ID NOS: 19 and 20, SEQ ID NOS: 21 and 22, SEQ ID NOS: 23 and 24, SEQ ID NOS: 25 and 26, SEQ ID NOS: 27 and 28, SEQ ID NOS: 29 and 30, SEQ ID NOS: 31 and 32, SEQ ID NOS: 33 and 34, SEQ ID NOS: 35 and 36, and SEQ ID NOS: 37 and 38.

11. The method according to claim 9, further comprising performing first amplification as a first stage by using a combination of SEQ ID NOS: 18 and 19 as a primer combination, and subsequently performing second amplification for a product of the amplification by using a combination of SEQ ID NOS: 19 and 20 as a primer combination.

12. An expression vector comprising the nucleic acid as defined in claim 1 or 3.

13. A cell which is transformed by introducing the nucleic acid as defined in claim 1 or 3 and which expresses a polypeptide encoded by the nucleic acid.

14. A method for producing the polypeptide, comprising culturing the cell as defined in claim 13 and recovering the polypeptide from a culture.

15. An antibody against the polypeptide as defined in claim 4.


SEQUENCE LISTING (52 sequences)

SEQ ID NO: 1 - DNA, 9496 bp, Virus
agtttggtttgagaggaaaaagaggggactcacgggaacacgcttgacggcttcaatgacgatactggatccatcatgtccgttcccccaattcctagtgagaacgccgcatcggtcggtagcggtataatggacgagtgggggaacgatccggacatccaccaccagtgtatcgccatcgttatccgtggacggtccctgtccaccccacatgatccagccggcatggaccaggacaagccccaggtcagagtcatcggtataaacatccgtaccccagatccccctcttctcccatccggcactaatctccccacgccctttgccaacaccttcacggatcttgaccggacacgaggatgcatcttcccatccaggtgctcctctcaatttccagttcttcccaaggaaccccccacctatgtggaccttgacgagtccaccctgtagatttcgagaacggtgacgagataaacgataatccgaaccggtatacgtctcggtagctacaacccgattcatttccgatttcgtcttgagccatgcctcgatcgcggtactcttgacatcgggatatttacgtgtggcctcggtcaaatgactgaagtcgcgtattcgaacgggtgatttaggtcggtcctcgtctttacgccttttcgacttgttggcatttcctcgcctagcatcgtcgacggcagcatccattcgaggtcgtttatcccttggagtgtattgataatcccttgaatccgtcttgtattcatacggacgtggttctcgcctaggggcgacttgctgcagagcatcatgcggagtctgcgctgcaggatgggcaggattaccgtcgaggtatcgcctgaattgatcccgattcgcttgatccatacgttggtttgcttctttgatacgtgctgaattgatctcggcttgtcgagcatatgccagtcgttcctgctccttttgcctctctttagcttccctagcttctcggacttccttttccctctgagcagccgcagcacgcacacgagccatacgagcttcttgttcatcctgaagtcgtttcctctctcgatcgaatcgatcttgctctgccgtcgtatgtgctctttgagcagcatggctagccgagttaccggtccatccaccaaacgatccatactcgaatcctccactgaatggcatgaacccgccgtatccgggatagttggaaccggggaaatctcgtccaggtggtcgtgctgctgacgaggtggcagctgttgcgggtttgggagcggcagaggcggcagaagcggatcgagggggttcacgagccgatgggaggggaggttgggaggtgttgggcgtcgacttggcatcacgagctcctccaaaggcggacgtaccgaaagcggatcgagcgccaggttgagatgttgcggatttgcctggactgagctggccggggcccaaagcagcaggatcggggcgtgttccagtccctccactgggcgtgcgacggtgagctacaccaggagcttgtggagccgttgctgatggtggacgttctcgttggggatcgaatggtgagggtaaggatcggccgccgatggcagcgagtcctggtaggggtagattcgggcgctggcttgagccggtagcggttgctgttggaggtagagcgggggagggtttgttgggaatcactggggatgccgcggatggtaaagagcgggcattcgtaggcgacggtggaagtgatggacgagggtatgggtttgccggtgtcccagtggatgcgcgtggtgatgtcgctgggttcgccttggccgctctatcctttctctcctgctcaatcctcgcctgttccctctctcgctcacgctgacgagccctttcacgttccctcagatcttgctctgataatcccgacccaccaaacatacttcgatagccgtaattgaatggggcttcgcgcttgggaagtgatgcgctggcagtcgccggtgtcgtggaagttgcggcggtagcaggagttgaggctggtgtcggtgccgttgtaccatgagggtggtgatggtgatggtggtgatggtggtgaggtgctcctgggtgtgaatgctggatcgtaggagaggcggtagccggtgtagcaggttttggttcgtgacgcgtgcttccgatcgtatatttgggaacgtctgccgtcagctccgacatctcgcctatagttaactcaccagcgactggaggtggatctctcgatccaaaaggactataactaggtctcaatccattgccgggaccacctaatgaagacgccgaattgttccccggtctagctccggtatcgccgagtcctgcacctgtcaggctggggctactgcttgctgggttcctgggtcgttggaggcctccaaaattggagaatgatctgatgatggtggcttcttcggcgcgaggtggtcccgaggtctgagggaggttagtgcccggtcgttgggggagtgggtcggttggtttcggctgggggtttgtcgaggcgtggggagaaggcatcgtggttgggcgttccggtggatcgtatgtctagttgtattatatgtcgccaagagatgagagtaggtattatgctatgtgaacatgtgatgtatgacaataaggatatgcgaattgatggttgaattgaaaggatgaagatgacacagtttggcgttgcaataagcgtagaagttggtccctgaaccctgaaagtgtgtggacttgtgtatgccctagattacccctgtgcctgagtagcagaatcctggtaaacccgtttaaccgaatgtggcacttttaataggcgtgactgctttctttcattcaacctccactgtcaacaaacaactcagctatacactcatactcatacactccttcgaccatacatattgctatacccacactcgttctgcctgccttacctggctgctaagtcaacaactcgaccacgacgcgtatcaaccatctcgaaacacccgccaacaccgcaatcacaaagtaaacaatagatccgaaacgtcacattctaccgactcttacaccatgtctcgtgaactcgtctcgtcgtcatcgtatccactttccaatcccaccccatcttccagcatgaaatccagccagtcacgtataccggtgaaagccaagagagcaccactaggcgaaagagtcgacaaccacgatcacaccactccccgtcagcacctcgtcaagtctgtcaaatcagtcatcagacctcggatcatctctacaaagtcgaccgctagtccatcaaagtcgtccacataccgaccatcacctcgagcggcagtacaaggttccccttcatcctcaatcgtttcatctccagcgggagcaaatgtctcgagacccggacatacaccccaccctcgccgctcatctgttggtctcgcattccttggacatgacgtttcaatgttgatggatactactcggctggattttgtgagcgatacggatgatagtgagggagatgagccattggacaatatgctcgtgaggaggggagtgatgggaccagggagattgatgacgccagccaatagtcaggaatctcaggtaagctcattgtgcctcttggagtctcagctgacatgtagcccaactctcagcagcatactctgaaggagccatctccacgtcgtttggctatacgattacctacacctccagcatctcaatcaggtaccgatgtcgaggaaaccatctctcgaacgcttgtccgaccgagaccagcacctacacctccacgtcgatctgctggcccttcgatcctgctgaatggaatggcatctccaacccaagatatctcacccaatccccgaagaaaaaagtctcccagagtggatgaagctggaccttcgcaacttcgatccccattcgagattgcccctactactacttcgcctcgtcatcccagggatagagtcgagattgtcattggccagccatcgcgtaaatcagtctcgccctcggccacgcccaaagctagcaaaccaaggaagagcagagtatccactaaaaccccggcttctcacccattatcacaatcaacctcttcccaaccctctcgagcgaaaagggcaccggcaacagcccctcaaccgaatagacgaaagtcaacaccagccactcgtaaaccctctccacctagaacgacagctaaacccaaattcgctaaaacacccaagcggctctcactcccaggtcgaggtcgtgctcggaaatcagtcactccagccgaattgaaagcgatgattggtttactgccggagccagtggagcagagtagtccctccgacgatcccctgcttttagttagcgacgacgaggaccacacacccgctatcagaccttcatctcattctgggaagagtacaggatcaagatcggctgctaggattatcgatttcgatgctgccattcaaggtcgctcgagcgtagaggaggtcgtgagggattacgaggtacctatccacgggtcggaaccggattatgatgtcgctcgggattatggatttgattcaggtgattcagatgaggaaatggggaatgatacatttgtgaatgtcgccggagctcgagcgaaggtgaatccggcggatcatcaggacgaggcaaccctagaagatgaggatgagaatggaagtcacctcagctcaagatcaaactcgccattcgcatttcaagaagcggaagtacgattctcttcacctgctctttcgctccgagccgccagcccagcgaaagaggattctcgactcgtctcttcgcccgtcatcgcctctgtcgcctcgcctgttccatcagtcgccgctagtctaccctcgcctttcctctcacgctcgacgaactcgcctcgattgagcagatcaaccactgctacgccggtcgttcattcccatggcctaccatcgcctgctccttcctcgcctccgcccacggagatggagccagaggtggcatcctcgcctcgttatgcccggtcccagacaggtatccctcgagctacagtggctgtaaaccaggagctcgagtccgctaccgtaccagcttctcccgcctacagcgaaaagttctctgttggtatgctctcagctccctcgtctccgttctctaccagatcacgcgataaatccgccacgcctcgattcacagaacaattatcagtacattcgcagggaatggatgaagaggaagtacaggatccatgtagtgctagtccactactcctacctggaacaccatccgcccttggtctctttatgcctgaagaagcggatgggatgtcgactccatactcccacattggggcggagcctgatactcctgccccaaactttgattcgccctcacggtcaagcatacatatggactcgccagctccagtcgaagctcggacatcgccaactctctttggttcacgggtcgcatcgccagcccgctccgttgttgctcaagtcgcttcgcccgtcagacctatggctactcaggtcgcatcacctgttcgttcttctgcttctcagatggattcgcctgctcgctctgtttctcgagctggatcgcctgccctttcttctgcttctcgaattggatcgcctgccagaccagtcactctacaggctgcatcgcctgctcgttccactacttcccaggcagctgagctgtccatgggctattctcaatcaccgagaagatctccggtctgtctacctcaagagatggatctacccgaagaaataccgaccgtcagcaccagctcgcccaggaggtccccggtctgcctcccttctcatgtggatgttgtggacaacgtcgaggagcagccattaccagtcgtcaacacccattcgtttaggaagtcgccactcgcctacgcccaatcacctcgaaggtctccagtctgcttacccgccgacatggagaatgccgacgaagatgaagctggggataaagagaacatggcgcctgttcaatcgactactccaatcttatcaccccctacactaaaatcgactcctccaatgtacgctttcactttctctccccgaatcgcatcccccctccgaatagcccataatcaggcatccgctctgaaacgagacagcatcgctcgaacctctcaatcacctataccagctgttgaatcattccatgagagatttgcagaggttgatgaatgtcccagtgcggatgtgacattagaggggaacagtgcggattggagtttgtagtgatgagggtttaccggaggtggatcgtgaggaacatggacttggcttggacctacagggggaagatcaagagaacgacgataacctgtctatatccttatcgattgctgaagaaggtgaagaggaggaggagagtgatctcgtcgattcttccatcgagcaggaagatgatctggagcttggagccgatcccacgacccagctagcggacgacattgtcgactcgaatgaggacgagcaagatgctgaagaagatgcggagggatctgaagaggaggaggggcaggaatcgaatgtgattgcgccaatcccgactttcacggacgaacgaaatgcagcagaggaaggggagagtacggattctgaagaagccccatccagcgacgacgagcaggaggacgagaccgctcaggaagaattggaggaggatatccctgcttcacctgcacctgtcaagatcgttctcaaactcatcaatcggggtattgtcaagatcgaaccgcctgcctctcctgcccgggccaatgaagcggacgcctctgaactccgatcgaccactccggcttatgatccacctcagcctatcgtcgccgctcctatgactcgatcacctgcgcctgaggctacgacagtgtaccccgccctacctactacacccatcaccgcgcctctctcgcctcttgtgtccaccactcccctcggaactcctccagccaagcaacgatcgcctgccctgcagcagagtctaccccaatcttccgactacagcactctgcaagctggcccctctactcgaccacgatcccgactctctcgtcagattcccgccagtcccgaacaagaggaaagtgattccatcagagtccgaactcctcgtagtctcggtcttgagcttgaggatgccgaggccgataatagtatgcgatctgtggtcgaggtttcatctcttgatcccaaagctgccgctcgagctgctgctatcttgaaacttgtaagcttttttgattctttgcatggccaagctgatatgcagaaccacgcatacattgaacatggcgatatttcgaaacttggcgaatcgactgctgctcgacatcaatccatgcactcggctcgactcgacaagctcgatgcgtcccgtcgcgaagacaagactgagctcttgtacgaagccgagctggaaattgtttcgactcgccgaagtcgatctcggtccatgtctcggttcagaggaatcagcgaggcacccactgaattgccattgccaggcgcatggaatggtactcccaaacggaaacgatctgctcatctacaaattgaagctcctaagacaagcaacacttggggtgtatctcaatggaagaaacttgaaaaggtctttagggccgagagggagatttgggtaacggagcgtaatgtcaaagcgatgcctggcgggtttattggttgggcaaggatgtctacttttggaccgcctgctgcggttgctgtcccgtgggatccgactcgggtggtggataggttcctggaggaacagggtatctcgaaaaaggagcaagtgggagattggtcaaggtacgtccatcttccgtgatcttgaagggagctgatgaatagggaaatgatctatacccgcgtcgaagctctcgagaggcatgcggaacatcgaaagattaaagaaacgtctcaagaagagagatcacccaagaaaccaaagactgcgcctgctcctccgtcgaccgttcggaagttgtttggatgggtcatgccctccgctaccaagtctgctcttcccgtcgctgccccagaaagcagtgtaaagaatatggacaagggcaaagggaaagagagggagggtcgacagacattcaaggatcgacttgaggctcttcaggatcaacccatgactcagctttcagagtcggtctcgatccctcgacctacgcccatcgccagctcgactcccaatacggcaccacctcatccttcttctcctgcttggacgaatgcttaccagcccatctcaaccgccaacgcccacgctagcggcagcactaccactcgcccaactccccgaactttatctgcgatcctagctgaatcgtcgtccaccacctccagccgaggacaactccattcatccttatctcaacgatctgcggctctcgatgcgctgttcggtggatctacctcgacatcaaccaggccggtcgtacagatcaaaagaagttcgagcgttaaagatatagtcaggggatttgaagattcgggggctttggctaaaagtaccggtgaggaagggctgaggagggttcagagtagaccgctttagaggttttgaataacgaattataacgaattttccttggatgtctttaatgtaaagtaataatataatgcatatgtgcttgtctgcataacttatttgatgatcgatctgcggacatgccaagccattccaaggcgccggtcgaccgaaccttcctctcctcatccgccggtacatattcagggatatgggaatcgcgtgatttgggacgatatgaccacatcgataaccaagctccactaactatcaaaccactgggtaaagtatgcagagttgtctctgattctgacttattgttgtactgacgatggctcaacctgttgatgttgaagtaggtaacaaagtggcggctccggtagagagggagaaggatgaggggactattcggactactaggtactttccatctaatcccatgacgagggactgatgagagtgatcagttgtcagagacgtatatgaggcaacagactggagacgatgatgttcttgtcaagaaggtatgtccctcttattctgagtgaagagtggacacagtgggggcagaaagagagctgagggcagatggtacatgttaaccgggcaatcaatgagattggatggacgaaataccatctcaagctattctgtgagtaccctcgtcctttcgtgtactaactatactgacatccagttttgtaagctgtaacgccctctcgctgatggaactgatgggacaggaatggattcgggtacgcagtggatagtatgctaggtaagcccactggctcggatgaagctgatgaggtagttgtatgccaatcgattgctcaacctgctattcggatggaatttgaggggaggggcgttacgcaattattcgctgtatcactctcttctgctgttggtc

SEQ ID NO: 2 - DNA, 2631 bp, Virus
cccaactctcagcagcatactctgaaggagccatctccacgtcgtttggctatacgattacctacacctccagcatctcaatcaggtaccgatgtcgaggaaaccatctctcgaacgcttgtccgaccgagaccagcacctacacctccacgtcgatctgctggcccttcgatcctgctgaatggaatggcatctccaacccaagatatctcacccaatccccgaagaaaaaagtctcccagagtggatgaagctggaccttcgcaacttcgatccccattcgagattgcccctactactacttcgcctcgtcatcccagggatagagtcgagattgtcattggccagccatcgcgtaaatcagtctcgccctcggccacgcccaaagctagcaaaccaaggaagagcagagtatccactaaaaccccggcttctcacccattatcacaatcaacctcttcccaaccctctcgagcgaaaagggcaccggcaacagcccctcaaccgaatagacgaaagtcaacaccagccactcgtaaaccctctccacctagaacgacagctaaacccaaattcgctaaaacacccaagcggctctcactcccaggtcgaggtcgtgctcggaaatcagtcactccagccgaattgaaagcgatgattggtttactgccggagccagtggagcagagtagtccctccgacgatcccctgcttttagttagcgacgacgaggaccacacacccgctatcagaccttcatctcattctgggaagagtacaggatcaagatcggctgctaggattatcgatttcgatgctgccattcaaggtcgctcgagcgtagaggaggtcgtgagggattacgaggtacctatccacgggtcggaaccggattatgatgtcgctcgggattatggatttgattcaggtgattcagatgaggaaatggggaatgatacatttgtgaatgtcgccggagctcgagcgaaggtgaatccggcggatcatcaggacgaggcaaccctagaagatgaggatgagaatggaagtcacctcagctcaagatcaaactcgccattcgcatttcaagaagcggaagtacgattctcttcacctgctctttcgctccgagccgccagcccagcgaaagaggattctcgactcgtctcttcgcccgtcatcgcctctgtcgcctcgcctgttccatcagtcgccgctagtctaccctcgcctttcctctcacgctcgacgaactcgcctcgattgagcagatcaaccactgctacgccggtcgttcattcccatggcctaccatcgcctgctccttcctcgcctccgcccacggagatggagccagaggtggcatcctcgcctcgttatgcccggtcccagacaggtatccctcgagctacagtggctgtaaaccaggagctcgagtccgctaccgtaccagcttctcccgcctacagcgaaaagttctctgttggtatgctctcagctccctcgtctccgttctctaccagatcacgcgataaatccgccacgcctcgattcacagaacaattatcagtacattcgcagggaatggatgaagaggaagtacaggatccatgtagtgctagtccactactcctacctggaacaccatccgcccttggtctctttatgcctgaagaagcggatgggatgtcgactccatactcccacattggggcggagcctgatactcctgccccaaactttgattcgccctcacggtcaagcatacatatggactcgccagctccagtcgaagctcggacatcgccaactctctttggttcacgggtcgcatcgccagcccgctccgttgttgctcaagtcgcttcgcccgtcagacctatggctactcaggtcgcatcacctgttcgttcttctgcttctcagatggattcgcctgctcgctctgtttctcgagctggatcgcctgccctttcttctgcttctcgaattggatcgcctgccagaccagtcactctacaggctgcatcgcctgctcgttccactacttcccaggcagctgagctgtccatgggctattctcaatcaccgagaagatctccggtctgtctacctcaagagatggatctacccgaagaaataccgaccgtcagcaccagctcgcccaggaggtccccggtctgcctcccttctcatgtggatgttgtggacaacgtcgaggagcagccattaccagtcgtcaacacccattcgtttaggaagtcgccactcgcctacgcccaatcacctcgaaggtctccagtctgcttacccgccgacatggagaatgccgacgaagatgaagctggggataaagagaacatggcgcctgttcaatcgactactccaatcttatcaccccctacactaaaatcgactcctccaatgtacgctttcactttctctccccgaatcgcatcccccctccgaatagcccataatcaggcatccgctctgaaacgagacagcatcgctcgaacctctcaatcacctataccagctgttgaatcattccatgagagatttgcagaggttgatgaatgtcccagtgcggatgtgacattagaggggaacagtgcggattggagtttg

SEQ ID NO: 3 - DNA, 435 bp, Virus
gaggaaaaagaggggactcacgggaacacgcttgacggcttcaatgacgatactggatccatcatgtccgttcccccaattcctagtgagaacgccgcatcggtcggtagcggtataatggacgagtgggggaacgatccggacatccaccaccagtgtatcgccatcgttatccgtggacggtccctgtccaccccacatgatccagccggcatggaccaggacaagccccaggtcagagtcatcggtataaacatccgtaccccagatccccctcttctcccatccggcactaatctccccacgccctttgccaacaccttcacggatcttgaccggacacgaggatgcatcttcccatccaggtgctcctctcaatttccagttcttcccaaggaaccccccacctatgtggaccttgacgagtccaccctg

SEQ ID NO: 4 - DNA, 861 bp, Virus
ccgaatgtggcacttttaataggcgtgactgctttctttcattcaacctccactgtcaacaaacaactcagctatacactcatactcatacactccttcgaccatacatattgctatacccacactcgttctgcctgccttacctggctgctaagtcaacaactcgaccacgacgcgtatcaaccatctcgaaacacccgccaacaccgcaatcacaaagtaaacaatagatccgaaacgtcacattctaccgactcttacaccatgtctcgtgaactcgtctcgtcgtcatcgtatccactttccaatcccaccccatcttccagcatgaaatccagccagtcacgtataccggtgaaagccaagagagcaccactaggcgaaagagtcgacaaccacgatcacaccactccccgtcagcacctcgtcaagtctgtcaaatcagtcatcagacctcggatcatctctacaaagtcgaccgctagtccatcaaagtcgtccacataccgaccatcacctcgagcggcagtacaaggttccccttcatcctcaatcgtttcatctccagcgggagcaaatgtctcgagacccggacatacaccccaccctcgccgctcatctgttggtctcgcattccttggacatgacgtttcaatgttgatggatactactcggctggattttgtgagcgatacggatgatagtgagggagatgagccattggacaatatgctcgtgaggaggggagtgatgggaccagggagattgatgacgccagccaatagtcaggaatctcaggtaagctcattgtgcctcttggagtctcagctgacatgtagcccaactctcagcagcatactc

SEQ ID NO: 5 - DNA, 984 bp, Virus
accaggagctcgagtccgctaccgtaccagcttctcccgcctacagcgaaaagttctctgttggtatgctctcagctccctcgtctccgttctctaccagatcacgcgataaatccgccacgcctcgattcacagaacaattatcagtacattcgcagggaatggatgaagaggaagtacaggatccatgtagtgctagtccactactcctacctggaacaccatccgcccttggtctctttatgcctgaagaagcggatgggatgtcgactccatactcccacattggggcggagcctgatactcctgccccaaactttgattcgccctcacggtcaagcatacatatggactcgccagctccagtcgaagctcggacatcgccaactctctttggttcacgggtcgcatcgccagcccgctccgttgttgctcaagtcgcttcgcccgtcagacctatggctactcaggtcgcatcacctgttcgttcttctgcttctcagatggattcgcctgctcgctctgtttctcgagctggatcgcctgccctttcttctgcttctcgaattggatcgcctgccagaccagtcactctacaggctgcatcgcctgctcgttccactacttcccaggcagctgagctgtccatgggctattctcaatcaccgagaagatctccggtctgtctacctcaagagatggatctacccgaagaaataccgaccgtcagcaccagctcgcccaggaggtccccggtctgcctcccttctcatgtggatgttgtggacaacgtcgaggagcagccattaccagtcgtcaacacccattcgtttaggaagtcgccactcgcctacgcccaatcacctcgaaggtctccagtctgcttacccgccgacatggagaatgccgacgaagatgaagctggggataaagagaacatggcgcctgttcaatcgactactccaatcttatcaccccctacac

SEQ ID NO: 6 - DNA, 1620 bp, Virus
aggggaacagtgcggattggagtttgtagtgatgagggtttaccggaggtggatcgtgaggaacatggacttggcttggacctacagggggaagatcaagagaacgacgataacctgtctatatccttatcgattgctgaagaaggtgaagaggaggaggagagtgatctcgtcgattcttccatcgagcaggaagatgatctggagcttggagccgatcccacgacccagctagcggacgacattgtcgactcgaatgaggacgagcaagatgctgaagaagatgcggagggatctgaagaggaggaggggcaggaatcgaatgtgattgcgccaatcccgactttcacggacgaacgaaatgcagcagaggaaggggagagtacggattctgaagaagccccatccagcgacgacgagcaggaggacgagaccgctcaggaagaattggaggaggatatccctgcttcacctgcacctgtcaagatcgttctcaaactcatcaatcggggtattgtcaagatcgaaccgcctgcctctcctgcccgggccaatgaagcggacgcctctgaactccgatcgaccactccggcttatgatccacctcagcctatcgtcgccgctcctatgactcgatcacctgcgcctgaggctacgacagtgtaccccgccctacctactacacccatcaccgcgcctctctcgcctcttgtgtccaccactcccctcggaactcctccagccaagcaacgatcgcctgccctgcagcagagtctaccccaatcttccgactacagcactctgcaagctggcccctctactcgaccacgatcccgactctctcgtcagattcccgccagtcccgaacaagaggaaagtgattccatcagagtccgaactcctcgtagtctcggtcttgagcttgaggatgccgaggccgataatagtatgcgatctgtggtcgaggtttcatctcttgatcccaaagctgccgctcgagctgctgctatcttgaaacttgtaagcttttttgattctttgcatggccaagctgatatgcagaaccacgcatacattgaacatggcgatatttcgaaacttggcgaatcgactgctgctcgacatcaatccatgcactcggctcgactcgacaagctcgatgcgtcccgtcgcgaagacaagactgagctcttgtacgaagccgagctggaaattgtttcgactcgccgaagtcgatctcggtccatgtctcggttcagaggaatcagcgaggcacccactgaattgccattgccaggcgcatggaatggtactcccaaacggaaacgatctgctcatctacaaattgaagctcctaagacaagcaacacttggggtgtatctcaatggaagaaacttgaaaaggtctttagggccgagagggagatttgggtaacggagcgtaatgtcaaagcgatgcctggcgggtttattggttgggcaaggatgtctacttttggaccgcctgctgcggttgctgtcccgtgggatccgactcgggtggtggataggttcctggaggaacagggtatctcgaaaaaggagcaagtgggagattggtcaaggtacgtccatcttccg

SEQ ID NO: 7 - DNA, 729 bp, Virus
agggagctgatgaatagggaaatgatctatacccgcgtcgaagctctcgagaggcatgcggaacatcgaaagattaaagaaacgtctcaagaagagagatcacccaagaaaccaaagactgcgcctgctcctccgtcgaccgttcggaagttgtttggatgggtcatgccctccgctaccaagtctgctcttcccgtcgctgccccagaaagcagtgtaaagaatatggacaagggcaaagggaaagagagggagggtcgacagacattcaaggatcgacttgaggctcttcaggatcaacccatgactcagctttcagagtcggtctcgatccctcgacctacgcccatcgccagctcgactcccaatacggcaccacctcatccttcttctcctgcttggacgaatgcttaccagcccatctcaaccgccaacgcccacgctagcggcagcactaccactcgcccaactccccgaactttatctgcgatcctagctgaatcgtcgtccaccacctccagccgaggacaactccattcatccttatctcaacgatctgcggctctcgatgcgctgttcggtggatctacctcgacatcaaccaggccggtcgtacagatcaaaagaagttcgagcgttaaagatatagtcaggggatttgaagattcgggggctttggctaaaagtaccggtgaggaagggctgaggagggttcagagtagaccgctt

SEQ ID NO: 8 - DNA, 2571 bp, Virus
acatacgatccaccggaacgcccaaccacgatgccttctccccacgcctcgacaaacccccagccgaaaccaaccgacccactcccccaacgaccgggcactaacctccctcagacctcgggaccacctcgcgccgaagaagccaccatcatcagatcattctccaattttggaggcctccaacgacccaggaacccagcaagcagtagccccagcctgacaggtgcaggactcggcgataccggagctagaccggggaacaattcggcgtcttcattaggtggtcccggcaatggattgagacctagttatagtccttttggatcgagagatccacctccagtcgctggtgagttaactataggcgagatgtcggagctgacggcagacgttcccaaatatacgatcggaagcacgcgtcacgaaccaaaacctgctacaccggctaccgcctctcctacgatccagcattcacacccaggagcacctcaccaccatcaccaccatcaccatcaccaccctcatggtacaacggcaccgacaccagcctcaactcctgctaccgccgcaacttccacgacaccggcgactgccagcgcatcacttcccaagcgcgaagccccattcaattacggctatcgaagtatgtttggtgggtcgggattatcagagcaagatctgagggaacgtgaaagggctcgtcagcgtgagcgagagagggaacaggcgaggattgagcaggagagaaaggatagagcggccaaggcgaacccagcgacatcaccacgcgcatccactgggacaccggcaaacccataccctcgtccatcacttccaccgtcgcctacgaatgcccgctctttaccatccgcggcatccccagtgattcccaacaaaccctcccccgctctacctccaacagcaaccgctaccggctcaagccagcgcccgaatctacccctaccaggactcgctgccatcggcggccgatccttaccctcaccattcgatccccaacgagaacgtccaccatcagcaacggctccacaagctcctggtgtagctcaccgtcgcacgcccagtggagggactggaacacgccccgatcctgctgctttgggccccggccagctcagtccaggcaaatccgcaacatctcaacctggcgctcgatccgctttcggtacgtccgcctttggaggagctcgtgatgccaagtcgacgcccaacacctcccaacctcccctcccatcggctcgtgaaccccctcgatccgcttctgccgcctctgccgctcccaaacccgcaacagctgccacctcgtcagcagcacgaccacctggacgagatttccccggttccaactatcccggatacggcgggttcatgccattcagtggaggattcgagtatggatcgtttggtggatggaccggtaactcggctagccatgctgctcaaagagcacatacgacggcagagcaagatcgattcgatcgagagaggaaacgacttcaggatgaacaagaagctcgtatggctcgtgtgcgtgctgcggctgctcagagggaaaaggaagtccgagaagctagggaagctaaagagaggcaaaaggagcaggaacgactggcatatgctcgacaagccgagatcaattcagcacgtatcaaagaagcaaaccaacgtatggatcaagcgaatcgggatcaattcaggcgatacctcgacggtaatcctgcccatcctgcagcgcagactccgcatgatgctctgcagcaagtcgcccctaggcgagaaccacgtccgtatgaatacaagacggattcaagggattatcaatacactccaagggataaacgacctcgaatggatgctgccgtcgacgatgctaggcgaggaaatgccaacaagtcgaaaaggcgtaaagacgaggaccgacctaaatcacccgttcgaatacgcgacttcagtcatttgaccgaggccacacgtaaatatcccgatgtcaagagtaccgcgatcgaggcatggctcaagacgaaatcggaaatgaatcgggttgtagctaccgagacgtataccggttcggattatcgtttatctcgtcaccgttctcgaaatctacagggtggactcgtcaaggtccacataggtggggggttccttgggaagaactggaaattgagaggagcacctggatgggaagatgcatcctcgtgtccggtcaagatccgtgaaggtgttggcaaagggcgtggggagattagtgccggatgggagaagagggggatctggggtacggatgtttataccgatgactctgacctggggcttgtcctggtccatgccggctggatcatgtggggtggacagggaccgtccacggataacgatggcgatacactggtggtggatgtccggatcgttcccccactcgtccattataccgctaccgaccgatgcggcgttctcactaggaattgggggaacggacatgatggatccagtatcgtcattgaagccgtcaagcgtgttcccgtgagtcccctctttttcctctcaaaccaaact

SEQ ID NO: 9 - DNA, 912 bp, Virus
ctcaccgtcgcacgcccagtggagggactggaacacgccccgatcctgctgctttgggccccggccagctcagtccaggcaaatccgcaacatctcaacctggcgctcgatccgctttcggtacgtccgcctttggaggagctcgtgatgccaagtcgacgcccaacacctcccaacctcccctcccatcggctcgtgaaccccctcgatccgcttctgccgcctctgccgctcccaaacccgcaacagctgccacctcgtcagcagcacgaccacctggacgagatttccccggttccaactatcccggatacggcgggttcatgccattcagtggaggattcgagtatggatcgtttggtggatggaccggtaactcggctagccatgctgctcaaagagcacatacgacggcagagcaagatcgattcgatcgagagaggaaacgacttcaggatgaacaagaagctcgtatggctcgtgtgcgtgctgcggctgctcagagggaaaaggaagtccgagaagctagggaagctaaagagaggcaaaaggagcaggaacgactggcatatgctcgacaagccgagatcaattcagcacgtatcaaagaagcaaaccaacgtatggatcaagcgaatcgggatcaattcaggcgatacctcgacggtaatcctgcccatcctgcagcgcagactccgcatgatgctctgcagcaagtcgcccctaggcgagaaccacgtccgtatgaatacaagacggattcaagggattatcaatacactccaagggataaacgacctcgaatggatgctgccgtcgacgatgctaggcgaggaaatgccaacaagtcgaaaaggcgtaaagacgaggaccgacctaaatcacccgttcgaatacgcgacttcagtcatt

SEQ ID NO: 10 - protein, 877 aa, Virus
PNSQQHTLKEPSPRRLAIRLPTPPASQSGTDVEETISRTLVRPRPAPTPPRRSAGPSILLNGMASPTQDISPNPRRKKSPRVDEAGPSQLRSPFEIAPTTTSPRHPRDRVEIVIGQPSRKSVSPSATPKASKPRKSRVSTKTPASHPLSQSTSSQPSRAKRAPATAPQPNRRKSTPATRKPSPPRTTAKPKFAKTPKRLSLPGRGRARKSVTPAELKAMIGLLPEPVEQSSPSDDPLLLVSDDEDHTPAIRPSSHSGKSTGSRSAARIIDFDAAIQGRSSVEEVVRDYEVPIHGSEPDYDVARDYGFDSGDSDEEMGNDTFVNVAGARAKVNPADHQDEATLEDEDENGSHLSSRSNSPFAFQEAEVRFSSPALSLRAASPAKEDSRLVSSPVIASVASPVPSVAASLPSPFLSRSTNSPRLSRSTTATPVVHSHGLPSPAPSSPPPTEMEPEVASSPRYARSQTGIPRATVAVNQELESATVPASPAYSEKFSVGMLSAPSSPFSTRSRDKSATPRFTEQLSVHSQGMDEEEVQDPCSASPLLLPGTPSALGLFMPEEADGMSTPYSHIGAEPDTPAPNFDSPSRSSIHMDSPAPVEARTSPTLFGSRVASPARSVVAQVASPVRPMATQVASPVRSSASQMDSPARSVSRAGSPALSSASRIGSPARPVTLQAASPARSTTSQAAELSMGYSQSPRRSPVCLPQEMDLPEEIPTVSTSSPRRSPVCLPSHVDVVDNVEEQPLPVVNTHSFRKSPLAYAQSPRRSPVCLPADMENADEDEAGDKENMAPVQSTTPILSPPTLKSTPPMYAFTFSPRIASPLRIAHNQASALKRDSIARTSQSPIPAVESFHERFAEVDECPSADVTLEGNSADWSL

SEQ ID NO: 11 - protein, 145 aa, Virus
EEKEGTHGNTLDGFNDDTGSIMSVPPIPSENAASVGSGIMDEWGNDPDIHHQCIAIVIRGRSLSTPHDPAGMDQDKPQVRVIGINIRTPDPPLLPSGTNLPTPFANTFTDLDRTRGCIFPSRCSSQFPVLPKEPPTYVDLDESTL

SEQ ID NO: 12 - protein, 287 aa, Virus
PNVALLIGVTAFFHSTSTVNKQLSYTLILIHSFDHTYCYTHTRSACLTWLLSQQLDHDAYQPSRNTRQHRNHKVNNRSETSHSTDSYTMSRELVSSSSYPLSNPTPSSSMKSSQSRIPVKAKRAPLGERVDNHDHTTPRQHLVKSVKSVIRPRIISTKSTASPSKSSTYRPSPRAAVQGSPSSSIVSSPAGANVSRPGHTPHPRRSSVGLAFLGHDVSMLMDTTRLDFVSDTDDSEGDEPLDNMLVRRGVMGPGRLMTPANSQESQVSSLCLLESQLTCSPTLSSIL

SEQ ID NO: 13 - protein, 328 aa, Virus
TRSSSPLPYQLLPPTAKSSLLVCSQLPRLRSLPDHAINPPRLDSQNNYQYIRREWMKRKYRIHVVLVHYSYLEHHPPLVSLCLKKRMGCRLHTPTLGRSLILLPQTLIRPHGQAYIWTRQLQSKLGHRQLSLVHGSHRQPAPLLLKSLRPSDLWLLRSHHLFVLLLLRWIRLLALFLELDRLPFLLLLELDRLPDQSLYRLHRLLVPLLPRQLSCPWAILNHREDLRSVYLKRWIYPKKYRPSAPARPGGPRSASLLMWMLWTTSRSSHYQSSTPIRLGSRHSPTPNHLEGLQSAYPPTWRMPTKMKLGIKRTWRLFNRLLQSYHPLH

SEQ ID NO: 14 - protein, 540 aa, Virus
RGTVRIGVCSDEGLPEVDREEHGLGLDLQGEDQENDDNLSISLSIAEEGEEEEESDLVDSSIEQEDDLELGADPTTQLADDIVDSNEDEQDAEEDAEGSEEEEGQESNVIAPIPTFTDERNAAEEGESTDSEEAPSSDDEQEDETAQEELEEDIPASPAPVKIVLKLINRGIVKIEPPASPARANEADASELRSTTPAYDPPQPIVAAPMTRSPAPEATTVYPALPTTPITAPLSPLVSTTPLGTPPAKQRSPALQQSLPQSSDYSTLQAGPSTRPRSRLSRQIPASPEQEESDSIRVRTPRSLGLELEDAEADNSMRSVVEVSSLDPKAAARAAAILKLVSFFDSLHGQADMQNHAYIEHGDISKLGESTAARHQSMHSARLDKLDASRREDKTELLYEAELEIVSTRRSRSRSMSRFRGISEAPTELPLPGAWNGTPKRKRSAHLQIEAPKTSNTWGVSQWKKLEKVFRAEREIWVTERNVKAMPGGFIGWARMSTFGPPAAVAVPWDPTRVVDRFLEEQGISKKEQVGDWSRYVHLP

SEQ ID NO: 15 - protein, 243 aa, Virus
RELMNREMIYTRVEALERHAEHRKIKETSQEERSPKKPKTAPAPPSTVRKLFGWVMPSATKSALPVAAPESSVKNMDKGKGKEREGRQTFKDRLEALQDQPMTQLSESVSIPRPTPIASSTPNTAPPHPSSPAWTNAYQPISTANAHASGSTTTRPTPRTLSAILAESSSTTSSRGQLHSSLSQRSAALDALFGGSTSTSTRPVVQIKRSSSVKDIVRGFEDSGALAKSTGEEGLRRVQSRPL

SEQ ID NO: 16 - protein, 857 aa, Virus
TYDPPERPTTMPSPHASTNPQPKPTDPLPQRPGTNLPQTSGPPRAEEATIIRSFSNFGGLQRPRNPASSSPSLTGAGLGDTGARPGNNSASSLGGPGNGLRPSYSPFGSRDPPPVAGELTIGEMSELTADVPKYTIGSTRHEPKPATPATASPTIQHSHPGAPHHHHHHHHHHPHGTTAPTPASTPATAATSTTPATASASLPKREAPFNYGYRSMFGGSGLSEQDLRERERARQREREREQARIEQERKDRAAKANPATSPRASTGTPANPYPRPSLPPSPTNARSLPSAASPVIPNKPSPALPPTATATGSSQRPNLPLPGLAAIGGRSLPSPFDPQRERPPSATAPQAPGVAHRRTPSGGTGTRPDPAALGPGQLSPGKSATSQPGARSAFGTSAFGGARDAKSTPNTSQPPLPSAREPPRSASAASAAPKPATAATSSAARPPGRDFPGSNYPGYGGFMPFSGGFEYGSFGGWTGNSASHAAQRAHTTAEQDRFDRERKRLQDEQEARMARVRAAAAQREKEVREAREAKERQKEQERLAYARQAEINSARIKEANQRMDQANRDQFRRYLDGNPAHPAAQTPHDALQQVAPRREPRPYEYKTDSRDYQYTPRDKRPRMDAAVDDARRGNANKSKRRKDEDRPKSPVRIRDFSHLTEATRKYPDVKSTAIEAWLKTKSEMNRVVATETYTGSDYRLSRHRSRNLQGGLVKVHIGGGFLGKNWKLRGAPGWEDASSCPVKIREGVGKGRGEISAGWEKRGIWGTDVYTDDSDLGLVLVHAGWIMWGGQGPSTDNDGDTLVVDVRIVPPLVHYTATDRCGVLTRNWGNGHDGSSIVIEAVKRVPVSPLFFLSNQT

SEQ ID NO: 17 - protein, 304 aa, Virus
LTVARPVEGLEHAPILLLWAPASSVQANPQHLNLALDPLSVRPPLEELVMPSRRPTPPNLPSHRLVNPLDPLLPPLPLPNPQQLPPRQQHDHLDEISPVPTIPDTAGSCHSVEDSSMDRLVDGPVTRLAMLLKEHIRRQSKIDSIERGNDFRMNKKLVWLVCVLRLLRGKRKSEKLGKLKRGKRSRNDWHMLDKPRSIQHVSKKQTNVWIKRIGINSGDTSTVILPILQRRLRMMLCSKSPLGENHVRMNTRRIQGIINTLQGINDLEWMLPSTMLGEEMPTSRKGVKTRTDLNHPFEYATSVI

SEQ ID NO: 18 - DNA, 20 bp, Artificial Sequence
  primer 101-C
caacaccgcaatcacaaagt

SEQ ID NO: 19 - DNA, 20 bp, Artificial Sequence
  primer N101-B
aacattgaaacgtcatgtcc

SEQ ID NO: 20 - DNA, 20 bp, Artificial Sequence
  primer KS-2
ctcgtctcgtcgtcatcgta

SEQ ID NO: 21 - DNA, 21 bp, Artificial Sequence
  primer N101-D
catttgctcccgctggagatg

SEQ ID NO: 22 - DNA, 23 bp, Artificial Sequence
  primer K-5
aacgatccggacatccaccacca

SEQ ID NO: 23 - DNA, 20 bp, Artificial Sequence
  primer KIV-21
gcgatcgaggcatggctcaa

SEQ ID NO: 24 - DNA, 20 bp, Artificial Sequence
  primer 101-1a
ctgcagagcatcatgcggag

SEQ ID NO: 25 - DNA, 20 bp, Artificial Sequence
  primer 101-T
tcgatccgctttcggtacgt

SEQ ID NO: 26 - DNA, 20 bp, Artificial Sequence
  primer X-2
acctaatgaagacgccgaat

SEQ ID NO: 27 - DNA, 20 bp, Artificial Sequence
  primer KS-1
gagacatggtgtaagagtcg

SEQ ID NO: 28 - DNA, 20 bp, Artificial Sequence
  primer 101-11
agtggatgaagctggacctt

SEQ ID NO: 29 - DNA, 20 bp, Artificial Sequence
  primer Op-3
gatcttgatcctgtactctt

SEQ ID NO: 30 - DNA, 20 bp, Artificial Sequence
  primer 101-21
gcgaaagaggattctcgact

SEQ ID NO: 31 - DNA, 20 bp, Artificial Sequence
  primer X-7
tgggagtatggagtcgacat

SEQ ID NO: 32 - DNA, 21 bp, Artificial Sequence
  primer 101-23
tacactaaaatcgactcctcc

SEQ ID NO: 33 - DNA, 20 bp, Artificial Sequence
  primer X-13
atgagtttgagaacgatctt

SEQ ID NO: 34 - DNA, 20 bp, Artificial Sequence
  primer 101-29
acctgcgcctgaggctacga

SEQ ID NO: 35 - DNA, 20 bp, Artificial Sequence
  primer X-17
gcagatcgtttccgtttggg

SEQ ID NO: 36 - DNA, 21 bp, Artificial Sequence
  primer 101-38
aacatcgaaagattaaagaaa

SEQ ID NO: 37 - DNA, 20 bp, Artificial Sequence
  primer 101-6R
cacgcgattcccatatccct

SEQ ID NO: 38 - DNA, 20 bp, Artificial Sequence
  primer KIV-14
cgtgtactaactatactgac

SEQ ID NO: 39 - DNA, 20 bp, Artificial Sequence
  primer KIV-16
ctcaaattccatccgaatag

SEQ ID NO: 40 - DNA, 23 bp, Artificial Sequence
  primer IA-3
ccnacnggnagnggnaaragcac

SEQ ID NO: 41 - DNA, 23 bp, Artificial Sequence
  primer IV-3
ctnccmgtgcgnccncgscgytg

SEQ ID NO: 42 - DNA, 19 bp, Artificial Sequence
  primer III R-2
ccnggrnngtngcngtrgc

SEQ ID NO: 43 - DNA, 56 bp, Artificial Sequence
  primer 1-1F
tgatctagaatgcatcatcatcatcatcattctcgtgaactcgtctcgtcgtcatc

SEQ ID NO: 44 - DNA, 56 bp, Artificial Sequence
  primer 1-2F
tgatctagaatgcatcatcatcatcatcatgaatggcatctccaacccaagatatc

SEQ ID NO: 45 - DNA, 57 bp, Artificial Sequence
  primer 1-3F
tgatctagaatgcatcatcatcatcatcataagaggaagtacaggatccatgtagtg

SEQ ID NO: 46 - DNA, 55 bp, Artificial Sequence
  primer 1-4F
tgatctagaatgcatcatcatcatcatcatcgatctgtggtcgaggtttcatctc

SEQ ID NO: 47 - DNA, 56 bp, Artificial Sequence
  primer 1-5F
tgatctagaatgcatcatcatcatcatcataatagggaaatgatctatacccgcgt

SEQ ID NO: 48 - DNA, 32 bp, Artificial Sequence
  primer 1-1R
tcatttaaatcagagtatgctgctgagagttg

SEQ ID NO: 49 - DNA, 30 bp, Artificial Sequence
  primer 1-2R
tcatttaaatcaattcggctggagtgactg

SEQ ID NO: 50 - DNA, 31 bp, Artificial Sequence
  primer 1-3R
tcatttaaactattcggaggggggatgcgat

SEQ ID NO: 51 - DNA, 32 bp, Artificial Sequence
  primer 1-4R
tcatttaaatcacggaagatggacgtaccttg

SEQ ID NO: 52 - DNA, 30 bp, Artificial Sequence
  primer 1-5R
tcatttaaactaaagcggtctactctgaac